SEQUENCE LISTING (25 sequences)

SEQ ID NO: 1 - protein, 302 aa, Aspergillus tubigensis
QASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQAITDKNTLIEVMKNHITTVMQHYKGKIYAWDVVNEIFNEDGSLRDSVFYQVIGEDYVRIAFETARAADPNAKLYINDYNLDSASYPKLTGMVSHVKKWIEAGIPIDGIGSQTHLSAGGGAGISGALNALAGAGTKEIAVTELDIAGASSTDYVEVVEACLDQPKCIGITVWGVADPDSWRSSSTPLLFDSNYNPKPAYTAIANAL

SEQ ID NO: 2 - protein, 386 aa, Geosmithia emersonii
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 3 - protein, 308 aa, Aspergillus tubigensis
EPIEPRQASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQSITDKNTLIEVMKNHITTVMQHYKGKIYAWDVVNEIFNEDGSLRDSVFYKVIGEDYVRIAFETARAADPNAKLYINDYNLDSASYPKLTGMVSHVKKWIAAGIPIDGIGSQTHLSAGGGAGISGALNALAGAGTKEIAVTELDIAGASSTDYVEVVEACLNQPKCIGITVWGVADPDSWRSSSTPLLFDSNYNPKPAYTAIANAL

SEQ ID NO: 4 - protein, 406 aa, Aspergillus aculeatus
MVGLLSITAALAATVLPNIVSAVGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 5 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 6 - protein, 191 aa, Trichoderma reesei
QCIQPGTGYNNGYFYSYWNDGHGGVTYCNGPGGQFSVNWSNSGNFVGGKGWQPGTKNRVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVSD

SEQ ID NO: 7 - protein, 214 aa, Bacillus subtilis
QTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 8 - protein, 316 aa, Geosmithia emersonii
APVKEKGIKKRASPFQWFGSNESGAEFGNNNIPGVEGTDYTFPNTSAIQILIDQGMNIFRVPFLMERMVPNQMTGPVDSAYFQGYSQVINYITSHGASAVIDPHNFGRYYNNIISSPSDFQTFWHTIASNFADNDNVIFDTNNEYHDMDESLVVQLNQAAIDGIRAAGATSQYIFVEGNSWTGAWTWTQVNDAMANLTDPQNKIVYEMHQYLDSDGSGTSDQCVNSTIGQDRVESATAWLKQNGKKAILGEYAGGANSVCETAVTGMLDYLANNTDVWTGAIWWAAGPWWGDYIFSMEPPSGIAYEQVLPLLQPYL

SEQ ID NO: 9 - protein, 441 aa, Bacillus subtilis
DDYSVVEEHGQLSISNGELVNERGEQVQLKGMSSHGLQWYGQFVNYESMKWLRDDWGITVFRAAMYTSSGGYIDDPSVKEKVKETVEAAIDLGIYVIIDWHILSDNDPNIYKEEAKDFFDEMSELYGDYPNVIYEIANEPNGSDVTWDNQIKPYAEEVIPVIRDNDPNNIVIVGTGTWSQDVHHAADNQLADPNVMYAFHFYAGTHGQNLRDQVDYALDQGAAIFVSEWGTSAATGDGGVFLDEAQVWIDFMDERNLSWANWSLTHKDESSAALMPGANPTGGWTEAELSPSGTFVREKIRESASIPPSDPTPPSDPGEPDPGEPDPTPPSDPGEYPAWDSNQIYTNEIVYHNGQLWQAKWWTQNQEPGDPYGPWEPLKSDPDSGEPDPTPPSDPGEYPAWDSNQIYTNEIVYHNGQLWQAKWWTQNQEPGDPYGPWEPLN

SEQ ID NO: 10 - protein, 397 aa, Trichoderma reesei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 11 - protein, 218 aa, Trichoderma reesei
QTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 12 - protein, 322 aa, Trichoderma reesei
HGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTNPYYAQCLN

SEQ ID NO: 13 - protein, 761 aa, Trichoderma reesei
AFSWKNVKLGGGGGFVPGIIFHPKTKGVAYARTDIGGLYRLNADDSWTAVTDGIADNAGWHNWGIDAVALDPQDDQKVYAAVGMYTNSWDPSNGAIIRSSDRGATWSFTNLPFKVGGNMPGRGAGERLAVDPANSNIIYFGARSGNGLWKSTDGGVTFSKVSSFTATGTYIPDPSDSNGYNSDKQGLMWVTFDSTSSTTGGATSRIFVGTADNITASVYVSTNAGSTWSAVPGQPGKYFPHKAKLQPAEKALYLTYSWWPDAQLFRSTDSGTTWSPIWAWASYPTETYYYSISTPKAPWIKNNFIDVTSESPSDGLIKRLGWMIESLEIDPTDSNHWLYGTGMTIFGGHDLTNWDTRHNVSIQSLADGIEEFSVQDLASAPGGSELLAAVGDDNGFTFASRNDLGTSPQTVWATPTWATSTSVDYAGNSVKSVVRVGNTAGTQQVAISSDGGATWSIDYAADTSMNGGTVAYSADGDTILWSTASSGVQRSQFQGSFASVSSLPAGAVIASDKKTNSVFYAGSGSTFYVSKDTGSSFTRGPKLGSAGTIRDIAAHPTTAGTLYVSTDVGIFRSTDSGTTFGQVSTALTNTYQIALGVGSGSNWNLYAFGTGPSGARLYASGDSGASWTDIQGSQGFGSIDSTKVAGSGSTAGQVYVGTNGRGVFYAQGTVGGGTGGTSSSTKQSSSSTSSASSSTTLRSSVVSTTRASTVTSSRTSSAAGPTGSGVAGHYAQCGGIGWTGPTQCVAPYVCQKQNDYYYQCV

SEQ ID NO: 14 - protein, 230 aa, Trichoderma reesei
HGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 15 - protein, 419 aa, Trichoderma reesei
GKIKYLGVAIPGIDFGCDIDGSCPTDTSSVPLLSYKGGDGAGQMKHFAEDDGLNVFRISATWQFVLNNTVDGKLDELNWGSYNKVVNACLETGAYCMIDMHNFARYNGGIIGQGGVSDDIFVDLWVQIAKYYEDNDKIIFGLMNEPHDLDIEIWAQTCQKVVTAIRKAGATSQMILLPGTNFASVETYVSTGSAEALGKITNPDGSTDLLYFDVHKYLDINNSGSHAECTTDNVDAFNDFADWLRQNKRQAIISETGASMEPSCMTAFCAQNKAISENSDVYIGFVGWGAGSFDTSYILTLTPLGKPGNYTDNKLMNECILDQFTLDEKYRPTPTSISTAAEETATATATSDGDAPSTTKPIFREETASPTPNAVTKPSPDTSDSSDDDKDSAASMSAQGLTGTVLFTVAALGYMLVAF

SEQ ID NO: 16 - protein, 499 aa, Bacillus subtilis
MKRSISIFITCLLITLLTMGGMIASPASAAGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 17 - protein, 185 aa, Artificial sequence
  Bacillus subtilis xylanase variant
ASTDYWQNWTFGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 18 - protein, 185 aa, Artificial sequence
  Bacillus subtilis xylanase variant
ASTDYWQNWTDGYGIVNAVNGSGGNYSVNWSNTGNFVVGKGWTTGSPFRTINYNAGVWAPNGNGYLTLYGWTRSPLIEYYVVDSWGTYRPTGTYKGTVYSDGGWYDIYTATRDNAPSIDGDFTTFTQYWSVRQSKRPTGSNATITFSNHVNAWRSHGMDLGSNWAYQVMATEGYLSSGSSNVTVW

SEQ ID NO: 19 - protein, 343 aa, AUREOBASIDIUM PULLULANS
TPTDYSTSSYSKNQGLAQAWTSKGRQYIGTALTIRDDPVEQGIIQSRTDFNSITPENAMKWESTEPQRNNFTFAGADAVADFADRYNKEMRCHTLVWHSQLPAWVSQGNFDNKTLISIMENHIKKVAGRYKNKCTHWDVVNEALNEDGTYRSSVFYNTIGEAFIPIAFRFAEKYAGSKTKLYYNDYNLEYGSAKALGAQRILKLVQSYGVQIDGVGLQAHLSSEATASTGGGVTPDVQTLTNVLKLYTDLGVEVAYTELDVRFTTPATDAKLKAQADAYARVVQSCINVKRCVGITVWGVSDKYSWIPGVFPTEGAALLWDENFNKKPAYSSVLKTIQSFRKS

SEQ ID NO: 20 - protein, 225 aa, THERMOMYCES LANUGINOSUS
AFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVGMVGFTPVALAALAATGAL

SEQ ID NO: 21 - protein, 310 aa, THERMOASCUS AURANTIACUS
SPILEERQAAQSVDQLIKARGKVYFGVATDQNRLTTGKNAAIIQADFGQVTPENSMKWDATEPSQGNFNFAGADYLVNWAQQNGKLIRGHTLVWHSQLPSWVSSITDKNTLTNVMKNHITTLMTRYKGKIRAWDVVNEAFNEDGSLRQTVFLNVIGEDYIPIAFQTARAADPNAKLYINDYNLDSASYPKTQAIVNRVKQWRAAGVPIDGIGSQTHLSAGQGAGVLQALPLLASAGTPEVAITELDVAGASPTDYVNVVNACLNVQSCVGITVWGVADPDSWRASTTPLLFDGNFNPKPAYNAIVQDLQQ

SEQ ID NO: 22 - protein, 295 aa, Streptomyces thermoviolaceus
DTITSNQTGTHNGYFYSFWTDAPGTVTMNTGAGGNYSTQWSNTGNFVAGKGWATGGRRTVTYSGTFNPSGNAYLALYGWSQNPLVEYYIVDNWGTYRPTGTYKGTVDSDGGTYDIYMTTRYNAPSIEGTKTFNQYWSVRQNKRTGGTITTGNHFDAWAAHGMPLGTFNYMILATEGYQSSGSSNITVGDSGGDNGGGGGGGGGGGNTGGCTATLSAGEQWSDRYNLNVSVSGSDNWTVTMRVPAPEKVMATWNVTASYPDAQTLVARPNGNGNNWGVTIQKNGSTTWPTVSCSVG

SEQ ID NO: 23 - protein, 296 aa, THERMOBIFIDA FUSCA
AVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNPGGGNPPGGGNPPGGGGCTATLSAGQQWNDRYNLNVNVSGSNNWTVTVNVPWPARIIATWNIHASYPDSQTLVARPNGNGNNWGMTIMHNGNWTWPTVSCSAN

SEQ ID NO: 24 - protein, 328 aa, FUSARIUM OXYSPORUM
MKLSSFLYTASLVAAIPTAIEPRQASDSINKLIKNKGKLYYGTITDPNLLGVAKDTAIIKADFGAVTPENSMKWDATEPSQGKFNFGSFDQVVNFAQQNGLKVRGHTVVWHSQLPQWVKNINDKATLTKVIENHVTNVVGRYKGKIYAWDVVNEIFDWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKAAYTAVVNALR

SEQ ID NO: 25 - protein, 305 aa, FUSARIUM VERTICILLOIDES
QAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

(19) United States
(12) Patent Application Publication
(10) Pub. No.: US 2015/0337247 A1
(43) Pub. Date: Nov. 26, 2015

(54) ENZYMES

(71) Applicants: Jens Frisbaek SORENSEN, Arhus (DK); Lone Brond MILLER, Viby J (DK); Zhenghong ZHANG, Baoshan District, Shanghai (CN); Zheyong YU, Hongkou District, Shanghai (CN); Ling HUA, Minhang District, Shanghai (CN)

(72) Inventors: Jens Frisbaek SORENSEN, Arhus (DK); Lone Brond MILLER, Viby J (DK); Zhenghong ZHANG, Baoshan District, Shanghai (CN); Zheyong YU, Hongkou District, Shanghai (CN); Ling HUA, Minhang District, Shanghai (CN)

(73) Assignee: DUPONT NUTRITION BIOSCIENCES APS, Copenhagen (DK)

(21) Appl. No.: 14/419,191

(22) PCT Filed: Aug. 3, 2012
(86) PCT No.: PCT/CN2012/079653

§ 371 Date: Jun. 12, 2015

(51) Int. Cl. C12C 5/00; C12C 7/04; C08B 30/12; C12N 9/24; C12N 9/42

(52) U.S. Cl. CPC C12C 5/006; C12N 9/2482; C12N 9/244; C08B 30/12; C12C 7/04

(57) ABSTRACT

New enzymes with improved properties and compositions comprising these enzymes suitable for use in the production of a food, feed, or malt beverage product, such as in a brewing process, are provided.

### FIELD OF THE INVENTION

The present invention relates to enzymes with improved properties and to compositions comprising these enzymes suitable for use in the production of a food, beverage (e.g. beer), feed, or biofuel, such as in a brewing process.

### BACKGROUND OF THE INVENTION

The use of enzymes in beer production is well known. Application of enzymes to the mashing step to improve mash filterability and increase extract yield is described in WO 97/42302.

WO2005118769 and WO2005059084 relates to a mashing and filtration step in a process for the production of beer, and to enzymatic compositions for use in such a process.

WO1999057325 relates to strains of Penicillium funiculosum, to new enzyme mixtures obtained from it and nucleic sequences thereto.

However, there is a need for improved enzymes as well as combination of enzymes useful in the productions of food and beverage products, such as in the mashing, cooking and filtration steps in the production of an alcoholic beverage, such as beer or whiskey.

### OBJECT OF THE INVENTION

It is an object of embodiments of the invention to provide enzymes suitable for the production of food and beverage products, such as in the production of an alcoholic or non-alcoholic beverage, such as a cereal- or malt-based beverage like beer or whiskey. The enzymes provided may have improved properties in relation to the use in brewing. These wide varieties of improved properties comprise e.g. improved temperature optimums, improved ratio in activity on soluble (WE-AX) to insoluble (WU-AX) arabinoxylan substrates, reduced total pressure built up during lautering and/or filtration steps of a brewing process, as well as increased filterability of enzyme treated material.

### SUMMARY OF THE INVENTION

It has been found by the present inventor(s) that one or more enzyme as well as certain combinations of enzymes have improved properties relative to known enzymes and enzyme combinations, particularly in relation to the use in a process of brewing, wherein starch containing material is treated with the one or more enzyme to produce a brewing mash.

So, in a first aspect the present invention relates to an enzyme exhibiting endo-1,4-β-xylanase activity, which enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25, or any functional fragment thereof.

As used herein “functional fragment” refers to a truncated version of an enzyme with essentially the same or at least a significant degree of enzyme activity as the non-truncated reference enzyme.

In a second aspect, the present invention relates to an enzyme exhibiting endo-1,3(4)-β-glucanase activity, which enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof.

In a third aspect the present invention relates to a DNA construct comprising a DNA sequence encoding an enzyme according to the invention.

In a further aspect the present invention relates to a recombinant expression vector comprising a DNA construct comprising a DNA sequence encoding an enzyme according to the invention.

In a further aspect the present invention relates to a cell that has been transformed with a DNA construct comprising a DNA sequence encoding an enzyme according to the invention.

In a further aspect the present invention relates to preparation comprising an enzyme, or a DNA construct, or a vector, or a cell according to the invention.

In a further aspect the present invention relates to composition comprising an enzyme exhibiting endo-1,4-β-xylanase activity according to the invention in combination with any one or more β-glucanase.

In a further aspect the present invention relates to a composition comprising an enzyme exhibiting endo-1,3(4)-β-glucanase activity according to the invention in combination with any one or more xylanase.

In a further aspect the present invention relates to the use of an enzyme according to the invention, or a preparation according to the invention, or a composition according to the invention in the production of a food, feed, or malt beverage product, such as beer or whiskey.

In a further aspect the present invention relates to the use of an enzyme according to the invention, or a preparation according to the invention, or a composition according to the invention, in the production of dough or baked products.

In a further aspect the present invention relates to the use of an enzyme according to the invention, or a preparation according to the invention, or a composition according to the invention, in the preparation of pulp or paper.

In a further aspect the present invention relates to the use of an enzyme according to the invention, or a preparation according to the invention, or a composition according to the invention, for the preparation of cereal components. In some embodiments the cereal is rye, wheat, or barley.

In a further aspect the present invention relates to the use of enzyme according to the invention, or a preparation according to the invention, or a composition according to the invention, in the production of beer or modification of by-products from a brewing process.

In a further aspect the present invention relates to the use of enzyme according to the invention, or a preparation according to the invention, or a composition according to the invention, in the production of wine or juice.

In a further aspect the present invention relates to the use of enzyme according to the invention, or a preparation according to the invention, or a composition according to the invention, in the production of a first—or second-generation biofuel, such as bioethanol.

In a further aspect the present invention relates to a method of altering filterability of a starch comprising material, said method comprising the step of treating said starch comprising material with enzyme, or a preparation, or a composition according to the invention.

In a further aspect the present invention relates to a method of reducing pressure built up during lautering in a brewing application, said method comprising the step of treating a brewing mash with enzyme, or a preparation, or a composition according to the invention.

In a further aspect the present invention relates to a method for the production of a food, feed, or beverage product, such as an alcoholic or non-alcoholic beverage, such as a cereal- or malt-based beverage like beer or whiskey, said method comprising the step of treating a starch comprising material with enzyme, or a preparation, or a composition according to the invention.

In a further aspect the present invention relates to a method for the production of a brewing mash, said method comprising the step of treating a starch comprising material with an enzyme, or a preparation, or a composition according to the invention.

In a further aspect the present invention relates to a method for the production of a first—or second-generation biofuel, such as bioethanol, said method comprising the step of treating a starch comprising material with an enzyme, or a preparation, or a composition according to the invention.

In a further aspect the present invention relates to a product obtained by a method according to the invention.

In a further aspect the present invention relates to a composition comprising the product obtained by a method according to the invention, such as wherein the product is in a range of 0.1%-99.9%.

### LEGENDS TO THE FIGURE

FIG. 1: Mashing profile used in lab scale and pilot scale brewing. Mashing was initiated by a 10 min mashing-in period after which the enzyme was added.

FIG. 2: Pilot scale Brewing application results from verification of the glucanase and xylanases screening. The B. sub glucanase S combined with the A. tub xylanases was tested against a blank and UltraFlo max. Data collected was the average flow (L/h), the total pressure build up over the lautering (mm WC, where 1 mm WC=9.80665 Pa) and the max pressure recorded during the lautering (mm WC).

FIG. 3: Xylanase functionality in brewing

FIG. 4: Flow—lautering applying various xylanase candidates

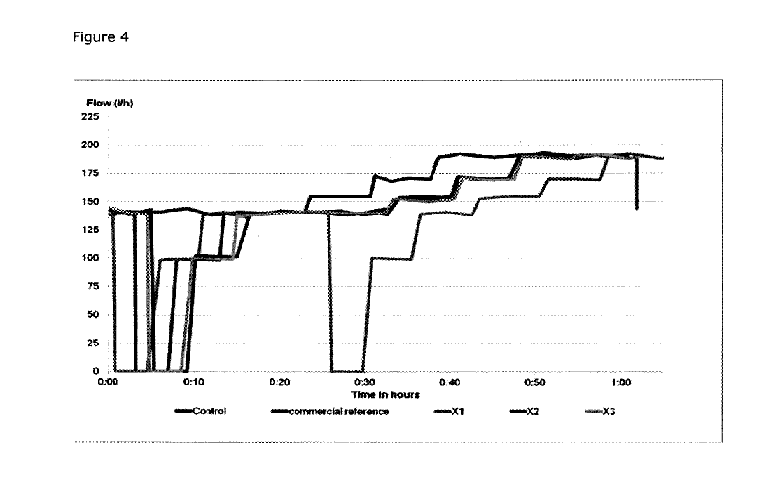

FIG. 5: Beer filtration—average of repeated filtrations

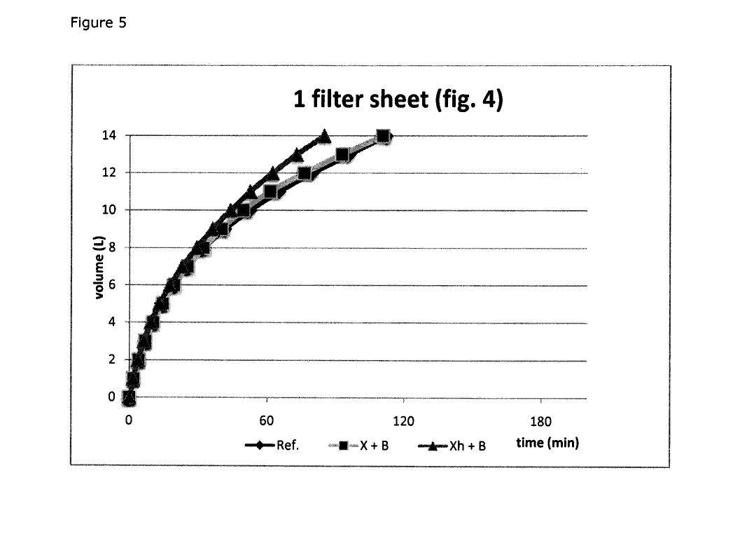

FIG. 6: Beer filtration—average of repeated filtrations

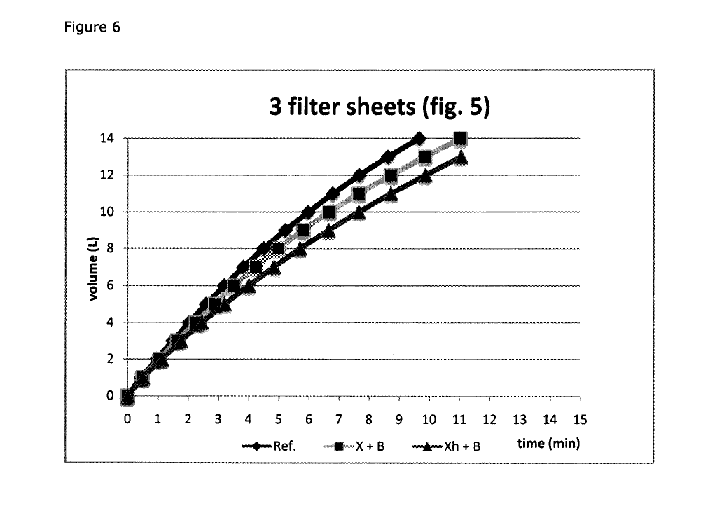

FIG. 7: Mashing diagram of example 3.

### DETAILED DISCLOSURE OF THE INVENTION

Beer is traditionally referred to as an alcoholic beverage derived from malt, such as malt derived from barley grain, and optionally adjunct, such as starch containing plant material (e.g. cereal grains) and optionally flavoured, e.g. with hops.

In the context of the present invention, the term “beer” is meant to comprise any fermented wort, produced by fermentation/brewing of a starch-containing plant material, thus in particular also beer produced exclusively from adjunct, or any combination of malt and adjunct.

The term “fermentation” means in the present context production of a substance such as ethanol by growing microorganisms in a culture. Commonly, microorganisms such as yeast are used for fermentation.

As used herein the term “malt” is understood as any malted cereal grain, such as malted barley. “Adjunct” can be defined as any starch-containing plant material which is not malt or barley malt.

“Starch-containing plant material” can e.g. be one or more cereal, such as barley, wheat, maize, rye, sorghum, millet, or rice, and any combination thereof. The starch-containing plant material can be processed, e.g. milled, malted, partially malted or unmalted. Unmalted cereal is also called “raw grain”. Examples of non-cereal starch-containing plant material comprise e.g. tubers, such as potatoes and cassava.

As used herein, the terms “beverage” and “beverage(s) product” includes such foam forming fermented beverages as full malted beer, beer brewed under the “Reinheitsgebot”, ale, dry beer, near beer, light beer, low alcohol beer, low calorie beer, porter, bock beer, stout, malt liquor, non-alcoholic beer, non-alcoholic malt liquor and the like. The term “beverages” or “beverages product” also includes non-foaming beer and alternative malt beverages such as fruit flavoured malt beverages, e. g., citrus flavoured, such as lemon-, orange-, lime-, or berry-flavoured malt beverages, liquor flavoured malt beverages, e. g., vodka-, rum-, or tequila-flavoured malt liquor, or coffee flavoured malt beverages, such as caffeine-flavoured malt liquor, and the like.

Beer can be made from a variety of starch-containing plant material by essentially the same process, where the starch is consists mainly of glucose homopolymers in which the glucose residues are linked by either alpha-1,4- or alpha-1,6-bonds, with the former predominating.

The process of making fermented beverages such as beer is commonly referred to as brewing. The traditional raw materials used in making these beverages are water, hops and malt. In addition or instead of malt, adjuncts such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch. The starch will eventually be converted enzymatically into fermentable sugars.

Concerning beers made predominantly from malt (e.g. up to 15-20% adjunct), for a number of reasons, the malt, which is produced principally from selected varieties of barley, has the greatest effect on the overall character and quality of the beer. First, the malt is the primary flavouring agent in beer. Second, the malt provides the major portion of the fermentable sugar. Third, the malt provides the proteins, which will contribute to the body and foam character of the beer. Fourth, the malt provides the necessary enzymatic activity during mashing.

Hops also contribute significantly to beer quality, including flavouring. In particular, hops (or hops constituents) add desirable bittering substances to the beer. In addition, the hops act as protein precipitants, establish preservative agents and aid in foam formation and stabilization. Not all beers are produced using hops. Other stabilizing agents, such as proteases (e.g. papain) may also be used.

### Without Wanting to be Construed as Limiting for the Present Invention, a Conventional Brewing Process can be Described as Follows:

The process for making beer is well known in the art, but briefly, it involves five steps: (a) mashing and/or adjunct cooking (b) wort separation and extraction (c) boiling and hopping of wort (d) cooling, fermentation and storage, and (e) maturation, processing and packaging. Typically, in the first step, milled or crushed malt is mixed with water and held for a period of time under controlled temperatures to permit the enzymes present in the malt to convert the starch present in the malt into fermentable sugars.

In the second step, the mash is transferred to a “lauter tun” or mash filter where the liquid is separated from the grain residue. This sweet liquid is called “wort” and the left over grain residue is called “spent grain”. The mash is typically subjected to an extraction, which involves adding water to the mash in order to recover the residual soluble extract from the spent grain.

In the third step, the wort is boiled vigorously. This sterilizes the wort and helps to develop the colour, flavour and odour. Hops are added at some point during the boiling.

In the fourth step, the wort is cooled and transferred to a fermentor, which either contains the yeast or to which yeast is added. The yeast converts the sugars by fermentation into alcohol and carbon dioxide gas; at the end of fermentation the fermentor is chilled or the fermentor may be chilled to stop fermentation. The yeast flocculates and is removed.

In the last step, the beer is cooled and stored for a period of time, during which the beer clarifies and its flavour develops, and any material that might impair the appearance, flavour and shelf life of the beer settles out. Prior to packaging, the beer is carbonated and, optionally, filtered and pasteurized.

After fermentation, a beverage is obtained which usually contains from about 2% to about 10% alcohol by weight. The non-fermentable carbohydrates are not converted during fermentation and form the majority of the dissolved solids in the final beer.

This residue remains because of the inability of malt amylases to hydrolyze the alpha-1,6-linkages of the starch. The non-fermentable carbohydrates contribute about 50 calories per 12 ounces of beer.

Recently, there has been a widespread popularization of brewed beverages called light beers, reduced calorie beers or low calorie beers, particularly in the U. S. market. As defined in the U.S., these beers have approximately 30% fewer calories than a manufacturers “normal” beer.

Further information on conventional brewing processes, as well as definitions for terms used in the field of brewing technology to be applied for the present invention, may be found in “Technology Brewing and Malting” by Wolfgang Kunze of the Research and Teaching Institute of Brewing, Berlin (VLB), 2nd revised Edition 1999, ISBN 3-921690-39-0, 3rd edition (2004): ISBN 3-921690-49-8, 4^{th} updated edition, 2010 (ISBN 978-3-921690-64-2).

Xylanases are classified in EC 3.2.1.8, EC 3.2.1.32, EC 3.2.1.136 and EC 3.2.1.156.; their activity may be measured e.g. as described in the examples. Suitable xylanases to be used in combination with an enzyme exhibiting endo-1,3(4)-β-glucanase activity according to the invention includes any xylanse classified in EC 3.2.1.8, EC 3.2.1.32, EC 3.2.1.136 and EC 3.2.1.156, such as any one disclosed in WO 2010072226, WO 2010072225, WO 2010072224, WO 2005059084, WO2007056321, WO2008023060A, WO9421785, WO2006114095, WO2006066582, US 2008233175, and WO10059424.

Endo-1,4-beta xylanase is classified as EC 3.2.1.8. The enzyme causes endohydrolysis of 1,4-beta-D-xylosidic linkages in xylans.

The terms “family 11 xylanase”, “Glycoside hydrolase (GH) family 11” or simply “GH 11 xylanase” as used herein refers to an endo-1,4-beta xylanase classified as EC 3.2.1.8, which causes endohydrolysis of 1,4-beta-D-xylosidic linkages in xylans and which is classified as a family 11 xylanase according to B. Henrissat, A classification of glycosyl hydrolases based on amino acid sequence similarities. Biochem. J. 280 (1991), pp. 309-316.

The terms “Family 10 xylanase”, “Glycoside hydrolase (GH) family 10”, or simply “GH 10 xylanse” comprises enzymes with a number of known activities, such as xylanase (EC:3.2.1.8); endo-1,3-beta-xylanase (EC:3.2.1.32); cellobiohydrolase (EC:3.2.1.91). These enzymes were formerly known as cellulase family F.

In some embodiments the enzyme exhibiting endo-1,4-β-xylanase activity is a family 11 xylanase. In some embodiments the enzyme exhibiting endo-1,4-β-xylanase activity is a family 10 xylanase.

In one aspect, the enzyme composition according to the invention has endo-1,4-beta xylanase activity as measured by the assay described in the examples.

An assay for measuring xylanase activity may be carried out at pH 3.5 or pH 5 and 50° C. using xylan as substrate, or it can be performed at different pH and temperature values for the additional characterisation and specification of enzymes. Enzyme activity is calculated from the increase in absorbance caused by xylose at 540 nm per unit time.

In some embodiments the enzyme composition according to the invention comprises a xylanase activity of at least about 5000 U/g, such as at least about 6000 U/g, such as at least about 7000 U/g, such as at least about 8000 U/g, such as at least about 8500 U/g, as measured by in the assay described in the examples.

The enzyme composition according to the invention may have cellulolytic activity. The systematic name of cellulose is 4-(1,3;1,4)-β-D-glucan 4-glucanohydrolase and cellulolytic enzymes or cellulases are classified in EC 3.2.1.4. Cellulase endohydrolyse (1→4)-β-D-glucosidic linkages in e.g. cellulose, lichenin and cereal β-D-glucans and will also hydrolyse 1,4-linkages in β-D-glucans also containing 1,3-linkages. Cellulase also have other names such as endo-1,4-β-D-glucanase, β-1,4-glucanase, β-1,4-endoglucan hydrolase, cellulase A, cellulosin AP, endoglucanase D, alkali cellulose, cellulase A 3, celludextrinase, 9.5 cellulase, avicelase, pancellase SS and 1,4-(1,3;1,4)-β-D-glucan 4-glucanohydrolase.

In one aspect of the invention, the cellulase activity of the enzyme composition according to the invention is measured by the “Cellulase activity method” as described in the following under the heading “Assays”.

In further aspects, the present invention relates to enzymes having endo-1,3(4)-β-glucanase activity is determined by the assay described in the examples.

“β-glucanase” or “beta-glucanase” as used herein refers to an endo-1,3(4)-beta-glucanase of EC 3.2.1.6. Catalyze the endohydrolysis of (1->3)- or (1->4)-linkages in beta-D-glucans when the glucose residue whose reducing group is involved in the linkage to be hydrolyzed is itself substituted at C-3. Suitable beta-glucanases to be used in combination with an enzyme exhibiting endo-1,4-β-xylanase activity according to the invention includes any one beta-glucanase disclosed in WO2004087889, WO2005059084, WO9414953, WO2007056321, WO9531533, WO08023060, WO2005100582, WO9828410, WO9742301, WO2006066582, WO05118769, WO2005003319, and WO10059424.

The standard assay is carried out at pH 5.0, and it can be performed at different pH values for the additional characterisation and specification of enzymes.

One unit of endo-1,3(4)-β-glucanase activity is defined as the amount of enzyme which produces 1 μmole glucose equivalents per minute under the conditions of the assay (pH 5.0 (or as specified) and 50° C.).

In some embodiments the enzyme composition according to the invention comprises a β-glucanase activity of at least about 10000 U/g, such as at least about 12000 U/g, such as at least about 14000 U/g, such as at least about 15000 U/g, such as at least about 18000 U/g as measured by the assay described in the examples.

In further aspects, the enzyme composition according to the invention has laminarinase activity or comprises any one or more further enzyme having laminarinase activity. The laminarinase activity is determined as described in the laminarase assay described in the Assay section.

Laminarinase may be an endo-1,3(4)-beta-glucanase classified in E.C. 3.2.1.6 or glucan endo-1,3-beta-D-glucosidase classified in E.C. 3.2.1.39. Endo-1,3(4)-beta-glucanase with the alternative names, laminarinase, endo-1,3-beta-glucanase, Endo-1,4-beta-glucanase is classified in E.C. 3.2.1.6. The substrates include laminarin, lichenin and cereal D-glucans and the enzyme catalyse endohydrolysis of (1->3)- or (1->4)-linkages in beta-D-glucans when the glucose residue whose reducing group is involved in the linkage to be hydrolyzed is itself substituted at C-3. Glucan endo-1,3-beta-D-glucosidase with the alternative names (1->3)-beta-glucan endohydrolase, Endo-1,3-beta-glucanase and laminarinase is classified in E.C. 3.2.1.39 and hydrolyse (1->3)-beta-D-glucosidic linkages in (1->3)-beta-D-glucans in substrates as eg. laminarin, paramylon and pachyman.

In some aspects, the enzyme composition according to the invention has arabinanase activity or comprises a further enzyme having arabinanase activity. Arabinanase is classified as EC 3.2.1.99. The systematic name is 5-α-L-arabinan 5-α-L-arabinanohydrolase but it has several other names such as arabinan endo-1,5-α-L-arabinosidase, and endo-1,5-α-L-arabinanase, endo-α-1,5-arabanase, endo-arabanase, 1,5-α-L-arabinan and 1,5-α-L-arabinanohydrolase. Arabinase endohydrolyses (1→5)-α-arabinofuranosidic linkages in (1→5)-arabinans. Arabinanase also acts on arabinan.

In one aspect of the invention, the arabinase activity of the enzyme composition according to the invention is measured by arabinase assay as described in the following under the heading “Assays”. The assay can be carried out at pH 3.5 and 50° C. using sugar beet arabinan as substrate, and it can be performed at different pH and temperature values for the additional characterisation and specification of enzymes. Enzyme activity is calculated from the increase in absorbance at 540 nm per unit time.

One unit of arabinase activity is defined as the amount of enzyme (normalised for total assay volume) that gives an increase in ΔOD_{540 nm}·min⁻¹ under the conditions of the assay (pH 3.5 and 50° C.).

In some aspects, the enzyme composition according to the invention has beta-D-glucoside glucohydrolase activity or comprises a further enzyme having beta-D-glucoside glucohydrolase activity. Beta-D-glucoside glucohydrolase refers to enzymes of E.C 3.2.1.21.

In some aspects, the enzyme composition according to the invention has β-Xylosidase activity or comprises a further enzyme having β-Xylosidase activity. “β-Xylosidase” or “Xylan 1,4-beta-xylosidase” refers to enzymes of E.C 3.2.1.37. β-Xylosidase catalyze the hydrolysis of (1->4)-beta-D-xylans, to remove successive D-xylose residues from the non-reducing termini.

In some aspects of the invention, the enzyme composition according to the invention has cellobiohydrolase activity or comprises a further enzyme having cellobiohydrolase activity. “Cellobiohydrolase” or “Cellulose 1,4-beta-cellobiosidase” refers to enzymes of EC 3.2.1.91. Cellulose 1,4-beta-cellobiosidase catalyze hydrolysis of 1,4-beta-D-glucosidic linkages in cellulose and cellotetraose, releasing cellobiose from the non-reducing ends of the chains.

The cellobiohydrolase activity of the enzyme composition according to the invention is measured by the cellobiohydrolase assay as described in the following under the heading “Assays”. The standard assay is carried out at pH 5.0, and it can be performed at different pH values for the additional characterisation and specification of enzymes.

One unit of cellobiohydrolase activity is defined as the amount of enzyme which produces 1 μmole p-nitrophenol from p-nitrophenyl β-D-cellobiopyranoside per minute under the conditions of the assay (pH 5.0 (or as specified) and 50° C.).

In some aspects, the enzyme composition according to the invention has α-N-arabinofuranosidase activity or comprises a further enzyme having arabinofuranosidase activity. “α-N-arabinofuranosidase” or “Alpha-N-arabinofuranosidase” refers to enzymes of EC 3.2.1.55. α-N-arabinofuranosidase catalyze the hydrolysis of terminal non-reducing alpha-L-arabinofuranoside residues in alpha-L-arabinosides.

In one aspect of the invention, the arabinofuranosidase activity of the enzyme composition according to the invention is measured by the arabinofuranosidase assay as described in the following under the heading “Assays”. The standard assay can be carried out at pH 5.0 and 50° C. and it can be performed at different values of pH and temperature for the additional characterisation and specification of enzymes.

One unit of α-N-arabinofuranosidase activity is defined as the amount of enzyme which produces 1 μmole p-nitrophenol from p-nitrophenyl α-L-arabinofuranoside per minute under the conditions of the assay (pH 5.0 and 50° C. (or as specified)).

In some aspects, the enzyme composition according to the invention has glucan 1,4-beta-glucosidase activity or comprises a further enzyme having glucan 1,4-beta-glucosidase activity. “Glucan 1,4-beta-glucosidase” or “glucan 1,4-beta-glucosidase” refers to enzymes of E.C3.2.1.74. Glucan 1,4-beta-glucosidase catalyze the hydrolysis of (1->4)-linkages in (1->4)-beta-D-glucans, to remove successive glucose units.

In some aspects, the enzyme composition according to the invention has xyloglucan-specific exo-beta-1,4-glucanase activity or comprises a further enzyme having xyloglucan-specific exo-beta-1,4-glucanase activity. “xyloglucan-specific exo-beta-1,4-glucanase” refers to enzymes of E.C3.2.1.155. Xyloglucan-specific exo-beta-1,4-glucanase catalyze the exohydrolysis of (1->4)-beta-D-glucosidic linkages in xyloglucan.

The enzymes and enzyme compositions according to the proceeding aspects may be used in a process comprising reducing the viscosity of an aqueous solution comprising a starch hydrolysate.

The enzymes and enzyme compositions may also be used in a process comprising filtering of an aqueous solution comprising a starch hydrolysate. In some embodiments the aqueous solution comprising a starch hydrolysate is a mash for beer making, and in other embodiments the aqueous solution comprising a starch hydrolysate is a food composition.

Alternatively, the enzyme composition according to the present invention may be used in the production of fruit juice, wine, grain processing, fuel alcohol, first—or second-generation biofuel, such as bioethanol, and potable alcohol.

In some embodiments the first—or second-generation biofuel, such as bioethanol is produced from agricultural feed stocks such as sugar cane, potato, corn, wheat sorghum etc. or from cellulosic material such as corn stover, switchgrass or other plant material. In both cases fermentable sugars are extracted from the raw material and fermented by microorganisms into alcohol, which is distilled and may be used as transportation fuel. The enzyme composition according to the present invention may be used in this production of biofuel. The enzymes complex may be added to enhance extraction of polysaccharides from the raw material, help degrade polysaccharides down into fermentable sugars and/or to enhance processing parameters such as separation of liquids from solids, flow characteristics and pumpability.

The process of the invention may be applied in the mashing of any grist. According to the invention the grist may comprise any starch and/or sugar containing plant material derivable from any plant and plant part, including tubers, roots, stems, leaves and seeds.

In some embodiments the grist comprises grain, such as grain from barley, wheat, rye, oat, corn, rice, milo, millet and sorghum, and more preferably, at least 10%, or more preferably at least 15%, even more preferably at least 25%, or most preferably at least 35%, such as at least 50%, at least 75%, at least 90% or even 100% (w/w) of the grist of the wort is derived from grain.

In some embodiments the grist comprises malted grain, such as barley malt. Preferably, at least 10%, or more preferably at least 15%, even more preferably at least 25%, or most preferably at least 35%, such as at least 50%, at least 75%, at least 90% or even 100% (w/w) of the grist of the wort is derived from malted grain.

The term “mash” is understood as aqueous starch slurry, e. g. comprising crushed barley malt, crushed barley, and/or other adjunct or a combination hereof, mixed with water later to be separated into wort+spent grains.

The term “mash separation” is understood as the separation of wort from spent grains, such as by lautering or mash filtration.

The term “beer filtration” is understood as a separation process in which the yeast cells and other turbidity-causing materials still present in the beer are removed, such as by microfiltration or membrane processes.

The enzyme preparation, such as in the form of a food ingredient prepared according to the present invention, may be in the form of a solution or as a solid—depending on the use and/or the mode of application and/or the mode of administration. The solid form can be either as a dried enzyme powder or as a granulated enzyme.

In one aspect the invention provides an enzyme composition preparation comprising the enzyme or enzyme composition according to the invention, an enzyme carrier and optionally a stabilizer and/or a preservative.

In yet a further aspect of the invention, the enzyme carrier is selected from the group consisting of glycerol or water.

In a further aspect, the preparation comprises a stabilizer. In one aspect, the stabilizer is selected from the group consisting of inorganic salts, polyols, sugars and combinations thereof. In one aspect, the stabilizer is an inorganic salt such as potassium chloride. In another aspect, the polyol is glycerol, propylene glycol, or sorbitol. In yet another aspect, the sugar is a small-molecule carbohydrate, in particular any of several sweet-tasting ones such as glucose, fructose and saccharose.

In yet at further aspect, the preparation comprises a preservative. In one aspect, the preservative is methyl paraben, propyl paraben, benzoate, sorbate or other food approved preservatives or a mixture thereof.

### SPECIFIC EMBODIMENTS OF THE INVENTION

In some embodiments the enzyme exhibiting endo-1,4-β-xylanase activity, optionally in combination with any one or more β-glucanase according to the present invention provides for a significantly reduced viscosity in brewing applications facilitating improved mash and beer separation.

Desired xylanase characteristics for brewing applications may include one or more of the following aspects:
a) Enzyme Substrate Specificity
   WE-AX/WU-AX ratio has an impact on viscosity. In some embodiments this ratio is less than about 7.0, such as less than about 6.5, such as less than about 6.0, such as less than about 5.5, such as less than about 5.0, such as less than about 4.5.
b) Enzyme Substrate Selectivity
   How close to branch points the enzyme(s) cutsis believed to have an impact on the functionality.
c) Enzyme Thermostability
   Continuous solubilisation of AX during mashing—thermostability a key feature. Accordingly, in some embodiments, the enzyme exhibiting endo-1,4-β-xylanase activity according to the present invention is thermostable within a temperature range of 65-78° C.
d) Enzyme pH optimum. Accordingly, in some embodiments, the enzyme exhibiting endo-1,4-β-xylanase activity has a pH optimum in the range of pH 5.4-5.6.
e) Enzyme inhibition (e.g. known key factor for xylanases).

Said significantly reduced viscosity in brewing applications may be measured as a reduced viscosity in the brewing application as compared to a control with a known enzyme or combination of enzyme activities, such as Ultraflo® Max used under same conditions and amounts.

In some embodiments, the enzyme exhibiting endo-1,4-β-xylanase activity according to the present invention, optionally in combination with any one or more β-glucanase according to the present invention provides for an improved mash and beer separation in brewing applications.

In some embodiments, the enzyme exhibiting endo-1,4-β-xylanase activity according to the present invention, optionally in combination with any one or more β-glucanase according to the present invention provides for a low potential for off flavour formation, such as off flavour formation related to arabinoxylan breakdown.

In some embodiments, the enzyme exhibiting endo-1,4-β-xylanase activity according to the present invention, optionally in combination with any one or more β-glucanase according to the present invention provides for a decreased risk of filter bed collapse, such asat lautering.

In some embodiments, the enzyme exhibiting endo-1,4-β-xylanase activity according to the present invention, optionally in combination with any one or more β-glucanase according to the present invention provides for a reduction in off flavour formation and/or off flavor potential.

One aspect of the invention relates to an enzyme exhibiting endo-1,4-β-xylanase activity, which enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25, or any functional fragment thereof.

Another aspect relates to an enzyme exhibiting endo-1,3(4)-β-glucanase activity, which enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof.

In some embodiments of the invention the enzyme exhibiting endo-1,4-β-xylanase activity has a ratio in activity on soluble arabinoxylan substrate (WE-AX) to insoluble arabinoxylan substrate (WU-AX) arabinoxylan substrate of less than about 7.0, such as less than about 6.5, such as less than about 6.0, such as less than about 5.5, such as less than about 5.0, such as less than about 4.5.

In some embodiments the enzyme according to the invention has a temperature optimum in the range of 40-70° C., such as in the range of 45-65° C., such as in the range of 50-65° C., such as in the range of 55-65° C.

In some embodiments the enzyme according to the invention has at least 81, 82, 83, 84, 85, 86, 87, 88, 89, 90, 91, 92, 93, 94, 95, 96, 97, 98 or 99% identity with any one amino acid sequence selected from SEQ ID NO: 1-25, or any functional fragment thereof.

In some embodiments the enzyme according to the invention has a total number of amino acids of less than 350, such as less than 340, such as less than 330, such as less than 320, such as less than 310, such as less than 300 amino acids, such as in the range of 200 to 350, such as in the range of 220 to 345 amino acids.

In some embodiments the amino acid sequence of said enzyme according to the invention has at least one, two, three, four, five, six, seven, eight, nine or ten amino acid substitutions as compared to any one amino acid sequence selected from SEQ ID NO: 1-25, or any functional fragment thereof.

In some embodiments the amino acid sequence of said enzyme according to the invention has a maximum of one, two, three, four, five, six, seven, eight, nine or ten amino acid substitutions compared to any one amino acid sequence selected from SEQ ID NO: 1-25, or any functional fragment thereof.

In some embodiments the enzyme according to the invention comprises the amino acid sequence identified by any one of SEQ ID NO: 1-25, or any functional fragment thereof.

In some embodiments the enzyme according to the invention consists of the amino acid sequence identified by any one of SEQ ID NO: 1-25, or any functional fragment thereof.

A further important aspect of the invention relates to a composition comprising an enzyme exhibiting endo-1,4-β-xylanase activity according to the invention in combination with any one or more β-glucanase. In some embodiments this one or more β-glucanase is according to the invention.

A further important aspect of the invention is a composition comprising an enzyme exhibiting endo-1,3(4)-β-glucanase activity according to the invention in combination with any one or more xylanase. In some embodiments this one or more xylanase is an enzyme exhibiting endo-1,4-β-xylanase activity according to the invention. In some embodiments this one or more xylanase is an enzyme according to SEQ ID NO:17 and/or SEQ ID NO:18. In some embodiments this one or more xylanase is an enzyme according to SEQ ID NO:19, SEQ ID NO:20, SEQ ID NO:21, SEQ ID NO:22, SEQ ID NO:23, SEQ ID NO:24, and/or SEQ ID NO:25, or any functional fragment thereof.

In some embodiments the combination of an enzyme exhibiting endo-1,4-β-xylanase activity with an enzyme exhibiting endo-1,3(4)-β-glucanase activity is according to the following table:

| | 1^{st} enzyme (Xylanase) | | | | | |
|---|---|---|---|---|---|---|
| 2^{nd} enzyme | SEQ ID | SEQ ID | SEQ ID | SEQ ID | SEQ ID | SEQ ID |
| (Glucanase) | NO: 1 | NO: 2 | NO: 3 | NO: 4 | NO: 5 | NO: 6 |
| SEQ ID NO: 7 | X | X | X | X | X | X |
| SEQ ID NO: 8 | X | X | X | X | X | X |
| SEQ ID NO: 9 | X | X | X | X | X | X |
| SEQ ID NO: 10 | X | X | X | X | X | X |
| SEQ ID NO: 11 | X | X | X | X | X | X |
| SEQ ID NO: 12 | X | X | X | X | X | X |
| SEQ ID NO: 13 | X | X | X | X | X | X |
| SEQ ID NO: 14 | X | X | X | X | X | X |
| SEQ ID NO: 15 | X | X | X | X | X | X |
| SEQ ID NO: 16 | X | X | X | X | X | X |

In some embodiments the combination of an enzyme exhibiting endo-1,4-β-xylanase activity with an enzyme exhibiting endo-1,3(4)-β-glucanase activity is according to the following table:

| | 1^{st} enzyme (Xylanase) | | | | | | | | |
|---|---|---|---|---|---|---|---|---|---|
| 2^{nd} enzyme | SEQ ID | SEQ ID | SEQ ID | SEQ ID | SEQ ID | SEQ ID | SEQ ID | SEQ ID | SEQ ID |
| (Glucanase) | NO: 17 | NO: 18 | NO: 19 | NO: 20 | NO: 21 | NO: 22 | NO: 23 | NO: 24 | NO: 25 |
| SEQ ID NO: 7 | X | X | X | X | X | X | X | X | X |
| SEQ ID NO: 8 | X | X | X | X | X | X | X | X | X |
| SEQ ID NO: 9 | X | X | X | X | X | X | X | X | X |
| SEQ ID NO: 10 | X | X | X | X | X | X | X | X | X |
| SEQ ID NO: 11 | X | X | X | X | X | X | X | X | X |
| SEQ ID NO: 12 | X | X | X | X | X | X | X | X | X |
| SEQ ID NO: 13 | X | X | X | X | X | X | X | X | X |
| SEQ ID NO: 14 | X | X | X | X | X | X | X | X | X |
| SEQ ID NO: 15 | X | X | X | X | X | X | X | X | X |
| SEQ ID NO: 16 | X | X | X | X | X | X | X | X | X |

It is to be understood that any one of the above combination of a 1^{st} enzyme being an enzyme exhibiting endo-1,4-β-xylanase activity may be combined with one one enzyme exhibiting endo-1,3(4)-β-glucanase activity with a ratio between the two enzymes of 1:10, 2:10, 3:10, 4:10, 5:10, 6:10, 7:10, 8:10, 9:10, 10:10, 10:9, 10:8, 10:7, 10:6, 10:5, 10:4, 10:3, 10:2, or 10:1, such as within a range of 1:10-10:1, such as 2:10-10:2, such as 3:10-10:3, such as 4:10-10:4, such as 5:10-10:5, such as 6:10-10:6, such as 7:10-10:7, such as 8:10-10:8, or within 9:10-10:9.

In some embodiments the composition according to the invention comprises a combination of at least two enzymes, said two enzymes, or two enzymes with an amino acid sequence having at least 80% sequence identity with the respective SEQ ID, or any functional fragment thereof, being selected from the list consisting of

SEQ ID NO:1 and SEQ ID NO:7; SEQ ID NO:2 and SEQ ID NO:7; SEQ ID NO:3 and SEQ ID NO:7; SEQ ID NO:4 and SEQ ID NO:7; SEQ ID NO:5 and SEQ ID NO:7; SEQ ID NO:6 and SEQ ID NO:7; SEQ ID NO:17 and SEQ ID NO:7; SEQ ID NO:18 and SEQ ID NO:7; SEQ ID NO:19 and SEQ ID NO:7; SEQ ID NO:20 and SEQ ID NO:7; SEQ ID NO:21 and SEQ ID NO:7; SEQ ID NO:22 and SEQ ID NO:7; SEQ ID NO:23 and SEQ ID NO:7; SEQ ID NO:24 and SEQ ID NO:7; SEQ ID NO:25 and SEQ ID NO:7; SEQ ID NO:1 and SEQ ID NO:8; SEQ ID NO:2 and SEQ ID NO:8; SEQ ID NO:3 and SEQ ID NO:8; SEQ ID NO:4 and SEQ ID NO:8; SEQ ID NO:5 and SEQ ID NO:8; SEQ ID NO:6 and SEQ ID NO:8; SEQ ID NO:17 and SEQ ID NO:8; SEQ ID NO:18 and SEQ ID NO:8; SEQ ID NO:19 and SEQ ID NO:8; SEQ ID NO:20 and SEQ ID NO:8; SEQ ID NO:21 and SEQ ID NO:8; SEQ ID NO:22 and SEQ ID NO:8; SEQ ID NO:23 and SEQ ID NO:8; SEQ ID NO:24 and SEQ ID NO:8; SEQ ID NO:25 and SEQ ID NO:8; SEQ ID NO:1 and SEQ ID NO:9; SEQ ID NO:2 and SEQ ID NO:9; SEQ ID NO:3 and SEQ ID NO:9; SEQ ID NO:4 and SEQ ID NO:9; SEQ ID NO:5 and SEQ ID NO:9; SEQ ID NO:6 and SEQ ID NO:9; SEQ ID NO:17 and SEQ ID NO:9; SEQ ID NO:18 and SEQ ID NO:9; SEQ ID NO:19 and SEQ ID NO:9; SEQ ID NO:20 and SEQ ID NO:9; SEQ ID NO:21 and SEQ ID NO:9; SEQ ID NO:22 and SEQ ID NO:9; SEQ ID NO:23 and SEQ ID NO:9; SEQ ID NO:24 and SEQ ID NO:9; SEQ ID NO:25 and SEQ ID NO:9; SEQ ID NO:1 and SEQ ID NO:10; SEQ ID NO:2 and SEQ ID NO:10; SEQ ID NO:3 and SEQ ID NO:10; SEQ ID NO:4 and SEQ ID NO:10; SEQ ID NO:5 and SEQ ID NO:10; SEQ ID NO:6 and SEQ ID NO:10; SEQ ID NO:17 and SEQ ID NO:10; SEQ ID NO:18 and SEQ ID NO:10; SEQ ID NO:19 and SEQ ID NO:10; SEQ ID NO:20 and SEQ ID NO:10; SEQ ID NO:21 and SEQ ID NO:10; SEQ ID NO:22 and SEQ ID NO:10; SEQ ID NO:23 and SEQ ID NO:10; SEQ ID NO:24 and SEQ ID NO:10; SEQ ID NO:25 and SEQ ID NO:10; SEQ ID NO:1 and SEQ ID NO:11; SEQ ID NO:2 and SEQ ID NO:11; SEQ ID NO:3 and SEQ ID NO:11; SEQ ID NO:4 and SEQ ID NO:11; SEQ ID NO:5 and SEQ ID NO:11; SEQ ID NO:6 and SEQ ID NO:11; SEQ ID NO:17 and SEQ ID NO:11; SEQ ID NO:18 and SEQ ID NO:11; SEQ ID NO:19 and SEQ ID NO:11; SEQ ID NO:20 and SEQ ID NO:11; SEQ ID NO:21 and SEQ ID NO:11; SEQ ID NO:22 and SEQ ID NO:11; SEQ ID NO:23 and SEQ ID NO:11; SEQ ID NO:24 and SEQ ID NO:11; SEQ ID NO:25 and SEQ ID NO:11; SEQ ID NO:1 and SEQ ID NO:12; SEQ ID NO:2 and SEQ ID NO:12; SEQ ID NO:3 and SEQ ID NO:12; SEQ ID NO:4 and SEQ ID NO:12; SEQ ID NO:5 and SEQ ID NO:12; SEQ ID NO:6 and SEQ ID NO:12; SEQ ID NO:17 and SEQ ID NO:12; SEQ ID NO:18 and SEQ ID NO:12; SEQ ID NO:19 and SEQ ID NO:12; SEQ ID NO:20 and SEQ ID NO:12; SEQ ID NO:21 and SEQ ID NO:12; SEQ ID NO:22 and SEQ ID NO:12; SEQ ID NO:23 and SEQ ID NO:12; SEQ ID NO:24 and SEQ ID NO:12; SEQ ID NO:25 and SEQ ID NO:12; SEQ ID NO:1 and SEQ ID NO:13; SEQ ID NO:2 and SEQ ID NO:13; SEQ ID NO:3 and SEQ ID NO:13; SEQ ID NO:4 and SEQ ID NO:13; SEQ ID NO:5 and SEQ ID NO:13; SEQ ID NO:6 and SEQ ID NO:13; SEQ ID NO:17 and SEQ ID NO:13; SEQ ID NO:18 and SEQ ID NO:13; SEQ ID NO:19 and SEQ ID NO:13; SEQ ID NO:20 and SEQ ID NO:13; SEQ ID NO:21 and SEQ ID NO:13; SEQ ID NO:22 and SEQ ID NO:13; SEQ ID NO:23 and SEQ ID NO:13; SEQ ID NO:24 and SEQ ID NO:13; SEQ ID NO:25 and SEQ ID NO:13; SEQ ID NO:1 and SEQ ID NO:14; SEQ ID NO:2 and SEQ ID NO:14; SEQ ID NO:3 and SEQ ID NO:14; SEQ ID NO:4 and SEQ ID NO:14; SEQ ID NO:5 and SEQ ID NO:14; SEQ ID NO:6 and SEQ ID NO:14; SEQ ID NO:17 and SEQ ID NO:14; SEQ ID NO:18 and SEQ ID NO:14; SEQ ID NO:19 and SEQ ID NO:14; SEQ ID NO:20 and SEQ ID NO:14; SEQ ID NO:21 and SEQ ID NO:14; SEQ ID NO:22 and SEQ ID NO:14; SEQ ID NO:23 and SEQ ID NO:14; SEQ ID NO:24 and SEQ ID NO:14; SEQ ID NO:25 and SEQ ID NO:14; SEQ ID NO:1 and SEQ ID NO:15; SEQ ID NO:2 and SEQ ID NO:15; SEQ ID NO:3 and SEQ ID NO:15; SEQ ID NO:4 and SEQ ID NO:15; SEQ ID NO:5 and SEQ ID NO:15; SEQ ID NO:6 and SEQ ID NO:15; SEQ ID NO:17 and SEQ ID NO:15; SEQ ID NO:18 and SEQ ID NO:15; SEQ ID NO:19 and SEQ ID NO:15; SEQ ID NO:20 and SEQ ID NO:15; SEQ ID NO:21 and SEQ ID NO:15; SEQ ID NO:22 and SEQ ID NO:15; SEQ ID NO:23 and SEQ ID NO:15; SEQ ID NO:24 and SEQ ID NO:15; SEQ ID NO:25 and SEQ ID NO:15; SEQ ID NO:1 and SEQ ID NO:16; SEQ ID NO:2 and SEQ ID NO:16; SEQ ID NO:3 and SEQ ID NO:16; SEQ ID NO:4 and SEQ ID NO:16; SEQ ID NO:5 and SEQ ID NO:16; SEQ ID NO:6 and SEQ ID NO:16; SEQ ID NO:17 and SEQ ID NO:16; SEQ ID NO:18 and SEQ ID NO:16; SEQ ID NO:19 and SEQ ID NO:16; SEQ ID NO:20 and SEQ ID NO:16; SEQ ID NO:21 and SEQ ID NO:16; SEQ ID NO:22 and SEQ ID NO:16; SEQ ID NO:23 and SEQ ID NO:16; SEQ ID NO:24 and SEQ ID NO:16; and SEQ ID NO:25 and SEQ ID NO:16.

In some embodiments the endo-1,3(4)-β-glucanase activity and the endo-1,4-β-xylanase activity are derived from at least two different enzymes, such as at least two different enzymes from two different species.

In some embodiments the total pressure built up is reduced to a value of less than 470 mm WC, such as less than 450 mm WC, such as less than 430 mm WC, such as less than 410 mm WC, such as less than 390 mm WC, such as less than 370 mm WC, such as less than 350 mm WC, such as less than 330 mm WC, such as less than 310 mm WC, such as less than 300 mm WC, such as less than 290 mm WC, when the composition according to the present invention is used prior to the lautering in a brewing application.

In some embodiments the total pressure built up is reduced by at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% compared to the use of a negative control without said composition; when used prior to the lautering in a brewing application.

In some embodiments the wort filterability as measured by volume wort collected after 5 min of filtration relative to a control without enzymes is increased to above 1.5, such as above 1.6, such as above 1.7, such as above 1.8, such as above 1.9, such as above 2.0, such as above 2.1, such as above 2.2, such as above 2.3, such as above 2.4, such as above 2.5, when the composition according to invention is used in a brewing application prior to the wort separation.

In some embodiments the wort filterability as measured by volume wort collected after 5 min of filtration is increased at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 110, 120, 130, 140, 150, 160, 170, 180, 190, 200, 210, 220, 230, 240, 250, 260, 270, 280, 290, or 300% as compared to the use of a negative control without said composition.

In some embodiments the composition according to the invention comprises any one or more further enzyme. In some embodiments the one or more further enzyme is selected from list consisting of a xylanase classified in EC 3.2.1.32, EC 3.2.1.136, or EC 3.2.1.156, a cellulase, a laminarinase, an endo-1,5-α-L-arabinanase, a beta-D-glucoside glucohydrolase, a β-Xylosidase, a cellobiohydrolase, a glucan 1,4-beta-glucosidase, a xyloglucan-specific exo-beta-1,4-glucanase and an α-N-Arabinofuranosidase.

Sequences and enzymes identified by a sequence as mentioned herein and used according to the present invention alone or in combinations with other enzymes or compounds may be with or without signal peptide.

### Assays

### DNS Cellulase Activity Method (DNS CMC Method)

Systematic Name: 1,4-(1,3;1,4)-β-D-glucan 4-glucanohydrolase

### IUB Number: EC 3.2.1.4

### Principle

The assay of cellulase is based on the enzymatic endo-hydrolysis of the 1,4-β-D-glucosidic bonds in carboxymethylcellulose (CMC), a β-1,4-glucan. The products of the reaction (β-1,4 glucan oligosaccharides) was determined colorimetrically by measuring the resulting increase in reducing groups using a 3,5-dinitrosalicylic acid reagent. Enzyme activity was calculated from the relationship between the concentration of reducing groups, as glucose equivalents, and absorbance at 540 nm.

The assay was carried out at pH 5.0, but it can be performed at different pH values for the additional characterisation and specification of enzymes.

### Unit Definition

One unit of cellulase activity is defined as the amount of enzyme which produces 1 μmole glucose equivalents per minute under the conditions of the assay (pH 5.0 (or as specified) and 50° C.).

### Materials

Carboxymethylcellulose. Supplier: Megazyme Ltd. Product no.: CM-Cellulose 4M

### D-Glucose ‘AnalaR’. Supplier: Merck Ltd (BDH). Product no.: 10117. M.W.: 180.16

Sodium acetate anhydrous ‘AnalaR’. Supplier: Merck Ltd (BDH). Product no.: 10236. M.W.: 82.03
Acetic acid (“glacial”) ‘AnalaR’. Supplier: Merck Ltd (BDH). Product no.: 10001. M.W.: 60.05
3,5-Dinitrosalicylic acid GPR (3,5-dinitro-2-hydroxybenzoic acid). Supplier: Merck Ltd (BDH). Product no.: 28235
Sodium hydroxide pellets ‘AnalaR’. Supplier: Merck Ltd (BDH). Product no.: 10252. M.W.: 40.00
Potassium sodium (+)-tartrate ‘AnalaR’. Supplier: Merck Ltd (BDH). Product no.: 10219. M.W.: 282.22
1.5% (w/v solution) Carboxymethylcellulose (CMC) solution in 0.1 M sodium acetate buffer, pH 5.0 (substrate solution).
3,5-Dinitrosalicylic acid (DNS) solution. 20 g/L of DNS in buffer containing 32 g/L sodium hydroxide pellets, and 600 g/L potassium sodium (+)-tartrate.
Glucose standard solution (0.50 mg/ml)

### Procedure

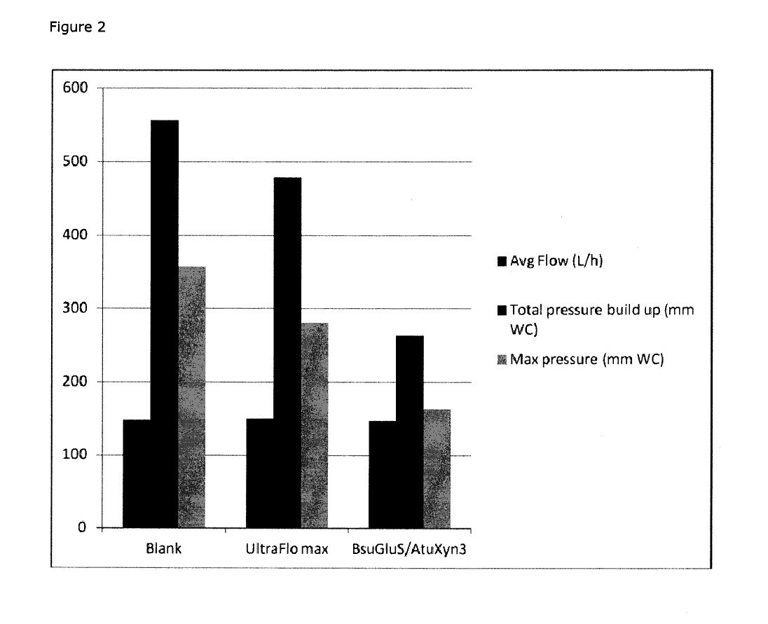

The enzyme composition was diluted into samples and a glucose standard curve as shown in FIG. 2 was made using glucose concentrations of 0, 0.125, 0.25, 0.375, and 0.5 mg/ml.

0.25 ml of enzyme solution was mixed with 1.75 ml of the substrate solution (1.5% w/v) at 50° C. and the reaction was stopped after 10 min by addition of DNS solution. This is followed by heating to 95° C. for 5 minutes.

The optical density was measured at 540 nm (OD_{540 nm}) of the different samples.

### Calculation

The enzyme activity is determined from the standard curve as shown in FIG. 2.

The activity is calculated as follows:

$$Activity ( u \cdot {ml}^{- 1} or u \cdot {g}^{- 1} ) = \frac{T - c}{m} \times A \times \frac{1}{180.16} \times {10}^{3} \times \frac{1}{V} \times \frac{1}{t} \times D$$

where:

### T=ΔOD_{540 nm} TEST

=OD_{540 nm} TEST−OD_{540 nm} BLANK
   m=gradient of the standard curve (approximately 1.0)
   c=y axis intercept of the standard curve (always negative and approximately −0.02)
   180.16≡molecular weight of glucose
   10³≡to convert to μmoles
   A=assay volume in ml
   V=enzyme volume in ml
   t=assay time in minutes
   D=actual enzyme dilution factor (e.g. for 1.000 g diluted to 1 litre D=1000)

### Laminarinase (DNS Laminarin Method)

### Principle

The reaction, catalysed by laminarinase, involves the endohydrolysis of 1,3-glucosidic bonds in 1,3-β-D-glucans. Substrates include laminarin, paramylon and pachyman. The products of the reaction (β-1,3-glucan oligosaccharides) are determined colourimetrically by measuring the resulting increase in reducing groups using a 3,5-dintrosalicylic acid reagent. Enzyme activity is calculated from the relationship between the concentration of reducing groups, as glucose equivalents, and absorbance at 540 nm.

The assay was carried out at pH 5.0 and 50° C., but it can be performed at different values of pH and temperature for the additional characterisation and specification of enzymes.

### Unit Definition

One unit of laminarinase activity is defined as the amount of enzyme which produces 1 μmole glucose equivalents per minute under the conditions of the assay (pH 5.0 and 50° C. (or as specified)).

### Materials

See materials given above for the Cellulase activity assay.

Laminarin (from Laminaria digitata). Supplier: Sigma-Aldrich Co. Ltd. Product no.: L 9634
1.00%(w/v solution) Laminarin solution (substrate solution 0.1M sodium acetate buffer, pH 5.0)
1.75 ml laminarin solution is mixed with 0.25 ml diluted enzyme solution at 50° C. for 10 minutes and the reaction stopped by addition of 2 ml DNS solution.
Standard curve was made using 0, 0.125, 0.25, 0.5 and 0.75 mg/ml glucose solution.
Optical density was measured at 540 nm (OD_{540 nm}).

### Calculation

The activity is calculated as follows:

$$Activity ( u \cdot {ml}^{- 1} or u \cdot {g}^{- 1} ) = \frac{T - c}{m} \times A \times \frac{1}{180.16} \times {10}^{3} \times \frac{1}{V} \times \frac{1}{t} \times D$$

where:

### T=ΔOD_{540 nm} TEST

=OD_{540 nm} TEST−OD_{540 nm} BLANK
   m=gradient of the standard curve (approximately 1.0)
   c=y axis intercept of the standard curve (always negative and approximately −0.03)
   180.16≡molecular weight of glucose
   10³≡to convert to μmoles
   A≡assay volume in ml
   V≡enzyme volume in ml
   t≡assay time in minutes
   D=enzyme dilution factor (e.g. for 1 g diluted to 1 litre D=1000)

### Arabinase Assay.

### Principle

The assay of Arabinase activity is based on colorimetrically determination by measuring the resulting increase in reducing groups using a 3,5-dinitrosalicylic acid reagent. Enzyme activity was calculated from the relationship between the concentration of reducing groups, as arabinose equivalents, and absorbance at 540 nm.

The assay was carried out at pH 3.5, but it can be performed at different pH values for the additional characterisation and specification of enzymes.

### Unit Definition

One unit of arabinase (Arabinanase (endo-1,5-alpha-L-arabinanase)) activity is defined as the amount of enzyme which produces 1 μmole arabinose equivalents per minute under the conditions of the assay (pH 3.5 (or as specified) and 50° C.).

### Materials

Megazyme Sugar Beet Arabinan

### Arabinose Sigma A3131 M.W.: 150.1

Sodium acetate anhydrous ‘AnalaR’. Supplier: Merck Ltd (BDH). Product no.: 10236. M.W.: 82.03
Acetic acid (“glacial”)′AnalaR′. Supplier: Merck Ltd (BDH). Product no.: 10001. M.W.: 60.05
3,5-Dinitrosalicylic acid GPR (3,5-dinitro-2-hydroxybenzoic acid). Supplier: Merck Ltd (BDH). Product no.: 28235
Sodium hydroxide pellets ‘AnalaR’. Supplier: Merck Ltd (BDH). Product no.: 10252. M.W.: 40.00
Potassium sodium (+)-tartrate ‘AnalaR’. Supplier: Merck Ltd (BDH). Product no.: 10219. M.W.: 282.22
1.5% (w/v solution) Arabinan solution in 0.1 M sodium acetate buffer, pH 3.5 (substrate solution).
3,5-Dinitrosalicylic acid (DNS) solution. 20 g/L of DNS in buffer containing 32 g/L sodium hydroxide pellets, and 600 g/L potassium sodium (+)-tartrate.
Arabinase standard solution (0.50 mg/ml)

### Procedure

The enzyme composition was diluted into samples and a glucose standard curve was made using arabinase concentrations of 0, 0.125, 0.25, 0.375, and 0.5 mg/ml.

0.25 ml of enzyme solution was mixed with 1.75 ml of the substrate solution (1.5% w/v) at 50° C. and the reaction was stopped after 10 min by addition of DNS solution. Followed by heating to 95° C. for 5 minutes.

The optical density was measured at 540 nm (OD_{540 nm}) of the different samples.

### Calculation

The enzyme activity is determined from the standard curve.

The activity is calculated as follows:

$$Activity ( u \cdot {ml}^{- 1} or u \cdot {g}^{- 1} ) = \frac{T - c}{m} \times A \times \frac{1}{150.13} \times {10}^{3} \times \frac{1}{V} \times \frac{1}{t} \times D$$

where:

### T=ΔOD_{540 nm} TEST

=OD_{540 nm} TEST−OD_{540 nm} BLANK
   m=gradient of the standard curve (approximately 1.0)
   c=y axis intercept of the standard curve (always negative and approximately −0.02)
   150.13≡molecular weight of arabinase
   10³≡to convert to μmoles
   A≡assay volume in ml
   V≡enzyme volume in ml
   t ≡assay time in minutes
   D=actual enzyme dilution factor (e.g. for 1.000 g diluted to 1 litre D=1000)

### Arabinofuranosidase Assay.

The reaction, catalysed by α-N-arabinofuranosidase, involves the hydrolysis of the terminal bond, at the non-reducing α-L-arabinofuranoside residue, of α-L-arabinosides. The enzyme acts on α-L-arabinofuranosides, α-L-arabinans containing (1,3)- and/or (1,5)-linkages, arabinoxylans and arabinogalactans.

The assay of α-N-arabinofuranosidase is based upon the enzymatic hydrolysis of p-nitrophenyl α-L-arabinofuranoside. The assay is a “two-point”, rather than a “continuous monitoring”, method. The calculation of enzyme activity is based on measurements taken only at the beginning and end of the incubation period. A product of the reaction, p-nitrophenol is determined colourimetrically (after pH adjustment). Enzyme activity is calculated from the relationship between the concentration of p-nitrophenol and absorbance at 400 nm.

### Preparation of Diluted Enzyme Solution:

Prepare all enzyme solutions, from powder or liquid enzyme preparations, with glass distilled water. Minimise assay dilution errors by avoiding large dilution steps involving small volumes or weights. In making enzyme dilutions it is more accurate, even for a liquid sample, to weigh out the initial enzyme sample. If this is done, in the case of liquid samples it is therefore necessary to measure the specific gravity of the liquid at 20° C.

As the assay is a “two-point”, rather than a “continuous monitoring”, method it is important to ensure the linearity within the incubation period with different enzyme systems and conditions. Under the standard assay conditions of substrate concentration, pH, temperature and assay time the assay has been demonstrated to be linear in the range ΔOD_{540 nm} TEST (T)=0.20-1.50. However, for good practice, the assay is operated within a defined range of ΔOD_{540 nm} TEST (T)=0.400-0.800.

### Procedure

Each enzyme sample assay involves three analyses: duplicate test (TEST) analyses and a blank (BLANK) analysis. The procedure given describes the analysis of a single enzyme sample.

| | TEST | BLANK |
|---|---|---|
| 0.2M Sodium acetate buffer, pH 5.0 | 1.00 ml | 1.00 ml |
| Glass distilled water | 1.00 ml | 1.00 ml |
| p-Nitrophenyl-α-L-arabinofuranoside solution | 1.00 ml | 1.00 ml |

0.25 ml diluted enzyme solution was added to the solutions at 50° C., the reaction was stopped after 10 minutes by addition of 4 ml of 0.4M glycine solution, pH 10.8 (stop reagent).

Absorbance was measured at 400 nm at 25° C. against a water blank.
determine OD_{400 nm} TEST for the duplicate TESTS measured;
determine OD_{400 nm} BLANK.

### Calculation

$$\begin{matrix}Δ {OD}_{400 nm} TEST ( T ) = {OD}_{400 nm} TEST - {OD}_{400 nm} BLANK \\ Units ( μ mol \cdot {min}^{- 1} ) = \frac{T}{18300} \times \frac{V}{1000} \times {10}^{6} \times \frac{1}{t}\end{matrix}$$

$$Activity ( u \cdot {ml}^{- 1} or u \cdot {g}^{- 1} ) = Units \times \frac{1}{E} \times D$$

where: T=OD400 nm TEST−OD400 nm BLANK
18300=Molar extinction coefficient for p-nitrophenol (1 cm path length)
V=7.25 (total liquid volume in test in ml)
t=10 (minutes)
1 u=1 μmol·min−1
E=0.25 (volume of diluted enzyme sample in ml)
D=Enzyme dilution factor e.g. for 1 ml diluted to 1 litre D=1000)

### Cellobiohydrolase Assay.

### Principle

The reaction, catalysed by cellobiohydrolase, involves the hydrolysis of 1,4-β-D-glucosidic linkages in cellulose and cellotetraose, releasing cellobiose from the non-reducing ends of the chains.

The assay of cellobiohydrolase is based on the enzymatic hydrolysis of p-nitrophenyl β-D-cellobiopyranoside. The product of the reaction, p-nitrophenol is determined colorimetrically (after pH adjustment). Enzyme activity is calculated from the relationship between the concentration of p-nitrophenol and absorbance at 400 nm.

The assay is operated within the linear defined range of ΔOD_{540 nm} TEST (T)=0.400-0.800.

### Procedure

Each enzyme sample assay involves three analyses: duplicate test (TEST) analyses and a blank (BLANK) analysis. The procedure given describes the analysis of a single enzyme sample.

| | TEST | BLANK |
|---|---|---|
| 0.2M Sodium acetate buffer, pH 5.0 | 1.00 ml | 1.00 ml |
| Glass distilled water | 1.00 ml | 1.00 ml |
| p-Nitrophenyl β-D-cellobiopyranoside solution | 1.00 ml | 1.00 ml |

0.25 ml diluted enzyme solution was added to the test solution at 50° C., after 30 minutes 4 ml of 0.4M glycine solution, pH 10.8 (stop reagent) was added to each tube.
   Absorbance was measured at 20° C. at 400 nm in a 1 cm glass cuvette against a water blank.
      determine OD400 nm TEST for the duplicate TESTS measured;
      determine OD400 nm BLANK.

### Calculation

$$\begin{matrix}Δ {OD}_{400 nm} TEST ( T ) = {OD}_{400 nm} TEST - {OD}_{400 nm} BLANK \\ Units ( μ mol \cdot {min}^{- 1} ) = \frac{T}{18300} \times \frac{V}{1000} \times {10}^{6} \times \frac{1}{t}\end{matrix}$$

$$Activity ( u \cdot {ml}^{- 1} or u \cdot {g}^{- 1} ) = Units \times \frac{1}{E} \times D$$

where: T=OD_{400 nm} TEST−OD_{400 nm} BLANK
18300=Molar extinction coefficient for p-nitrophenol (1 cm path length)
V=7.25 (total liquid volume in test in ml)
1000=to convert to litres
10⁶=to convert to μmoles
t=30 (minutes)
1 u=1 μmol·min⁻¹
E=0.25 (volume of diluted enzyme sample in ml)
D=Enzyme dilution factor e.g. for 1 ml diluted to 1 litre D=1000)

### Numbered Embodiments of the Invention

1. An enzyme exhibiting endo-1,4-β-xylanase activity, which enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25, or any functional fragment thereof.

2. The enzyme according to embodiment 1, which enzyme has a ratio in activity on soluble arabinoxylan substrate (WE-AX) to insoluble arabinoxylan substrate (WU-AX) arabinoxylan substrate of less than about 7.0, such as less than about 6.5, such as less than about 6.0, such as less than about 5.5, such as less than about 5.0, such as less than about 4.5.

3. An enzyme exhibiting endo-1,3(4)-β-glucanase activity, which enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof.

4. The enzyme according to any one of embodiment 1-3, which enzyme has a temperature optimum in the range of 40-70° C., such as in the range of 45-65° C., such as in the range of 50-65° C., such as in the range of 55-65° C.

5. The enzyme according to any one of embodiments 1-4, wherein said enzyme has at least 81, 82, 83, 84, 85, 86, 87, 88, 89, 90, 91, 92, 93, 94, 95, 96, 97, 98 or 99% identity with any one amino acid sequence selected from SEQ ID NO: 1-25, or any functional fragment thereof.

6. The enzyme according to any one of embodiments 1-5 having a total number of amino acids of less than 350, such as less than 340, such as less than 330, such as less than 320, such as less than 310, such as less than 300 amino acids, such as in the range of 200 to 350, such as in the range of 220 to 345 amino acids.

7. The enzyme according to any one of embodiments 1-6, wherein the amino acid sequence of said enzyme has at least one, two, three, four, five, six, seven, eight, nine or ten amino acid substitutions as compared to any one amino acid sequence selected from SEQ ID NO: 1-25, or any functional fragment thereof.

8. The enzyme according to any one of embodiments 1-7, wherein the amino acid sequence of said enzyme has a maximum of one, two, three, four, five, six, seven, eight, nine or ten amino acid substitutions compared to any one amino acid sequence selected from SEQ ID NO: 1-25, or any functional fragment thereof.

9. The enzyme according to any one of embodiments 1-8, which enzyme comprises the amino acid sequence identified by any one of SEQ ID NO: 1-25, or any functional fragment thereof.

10. The enzyme according to any one of embodiments 1 or 3, which enzyme consists of the amino acid sequence identified by any one of SEQ ID NO: 1-25, or any functional fragment thereof.

11. A DNA construct comprising a DNA sequence encoding an enzyme according to any of embodiments 1-10.

12. A recombinant expression vector comprising a DNA construct according to embodiment 11.

13. A cell that has been transformed with a DNA construct of embodiment 11 or the vector of embodiment 12.

14. A preparation comprising an enzyme according to any one of embodiments 1-10, or a DNA construct according to embodiment 11, or a vector according to embodiment 12, or a cell according to embodiment 13.

15. A composition comprising an enzyme exhibiting endo-1,4-β-xylanase activity according to any one of embodiments 1, 2, 4-10 in combination with any one or more β-glucanase.

16. The composition according to embodiment 15, wherein said one or more β-glucanase is an enzyme exhibiting endo-1,3(4)-β-glucanase activity according to any one of embodiments 3-10.

17. A composition comprising an enzyme exhibiting endo-1,3(4)-β-glucanase activity according to any one of embodiments 3-10 in combination with any one or more xylanase.

18. The composition according to embodiment 17, wherein said one or more xylanase is an enzyme exhibiting endo-1,4-β-xylanase activity according to any one of embodiments 1, 2, 4-10.

19. The composition according to any one of embodiments 15-18, wherein said endo-1,3(4)-β-glucanase activity and said endo-1,4-β-xylanase activity are derived from at least two different enzymes, such as at least two different enzymes from two different species.

20. The composition according to any one of embodiments 15-19, comprising a combination of at least two enzymes, said two enzymes, or two enzymes with an amino acid sequence having at least 80% sequence identity with the respective SEQ ID, or any functional fragment thereof, being selected from the list consisting of

### SEQ ID NO:1 and SEQ ID NO:7;

### SEQ ID NO:2 and SEQ ID NO:7;

### SEQ ID NO:3 and SEQ ID NO:7;

### SEQ ID NO:4 and SEQ ID NO:7;

### SEQ ID NO:5 and SEQ ID NO:7;

### SEQ ID NO:6 and SEQ ID NO:7;

### SEQ ID NO:17 and SEQ ID NO:7;

### SEQ ID NO:18 and SEQ ID NO:7;

### SEQ ID NO:19 and SEQ ID NO:7;

### SEQ ID NO:20 and SEQ ID NO:7;

### SEQ ID NO:21 and SEQ ID NO:7;

### SEQ ID NO:22 and SEQ ID NO:7;

### SEQ ID NO:23 and SEQ ID NO:7;

### SEQ ID NO:24 and SEQ ID NO:7;

### SEQ ID NO:25 and SEQ ID NO:7;

### SEQ ID NO:1 and SEQ ID NO:8;

### SEQ ID NO:2 and SEQ ID NO:8;

### SEQ ID NO:3 and SEQ ID NO:8;

### SEQ ID NO:4 and SEQ ID NO:8;

### SEQ ID NO:5 and SEQ ID NO:8;

### SEQ ID NO:6 and SEQ ID NO:8;

### SEQ ID NO:17 and SEQ ID NO:8;

### SEQ ID NO:18 and SEQ ID NO:8;

### SEQ ID NO:19 and SEQ ID NO:8;

### SEQ ID NO:20 and SEQ ID NO:8;

### SEQ ID NO:21 and SEQ ID NO:8;

### SEQ ID NO:22 and SEQ ID NO:8;

### SEQ ID NO:23 and SEQ ID NO:8;

### SEQ ID NO:24 and SEQ ID NO:8;

### SEQ ID NO:25 and SEQ ID NO:8;

### SEQ ID NO:1 and SEQ ID NO:9;

### SEQ ID NO:2 and SEQ ID NO:9;

### SEQ ID NO:3 and SEQ ID NO:9;

### SEQ ID NO:4 and SEQ ID NO:9;

### SEQ ID NO:5 and SEQ ID NO:9;

### SEQ ID NO:6 and SEQ ID NO:9;

### SEQ ID NO:17 and SEQ ID NO:9;

### SEQ ID NO:18 and SEQ ID NO:9;

### SEQ ID NO:19 and SEQ ID NO:9;

### SEQ ID NO:20 and SEQ ID NO:9;

### SEQ ID NO:21 and SEQ ID NO:9;

### SEQ ID NO:22 and SEQ ID NO:9;

### SEQ ID NO:23 and SEQ ID NO:9;

### SEQ ID NO:24 and SEQ ID NO:9;

### SEQ ID NO:25 and SEQ ID NO:9;

### SEQ ID NO:1 and SEQ ID NO:10;

### SEQ ID NO:2 and SEQ ID NO:10;

### SEQ ID NO:3 and SEQ ID NO:10;

### SEQ ID NO:4 and SEQ ID NO:10;

### SEQ ID NO:5 and SEQ ID NO:10;

### SEQ ID NO:6 and SEQ ID NO:10;

### SEQ ID NO:17 and SEQ ID NO:10;

### SEQ ID NO:18 and SEQ ID NO:10;

### SEQ ID NO:19 and SEQ ID NO:10;

### SEQ ID NO:20 and SEQ ID NO:10;

### SEQ ID NO:21 and SEQ ID NO:10;

### SEQ ID NO:22 and SEQ ID NO:10;

### SEQ ID NO:23 and SEQ ID NO:10;

### SEQ ID NO:24 and SEQ ID NO:10;

### SEQ ID NO:25 and SEQ ID NO:10;

### SEQ ID NO:1 and SEQ ID NO:11;

### SEQ ID NO:2 and SEQ ID NO:11;

### SEQ ID NO:3 and SEQ ID NO:11;

### SEQ ID NO:4 and SEQ ID NO:11;

### SEQ ID NO:5 and SEQ ID NO:11;

### SEQ ID NO:6 and SEQ ID NO:11;

### SEQ ID NO:17 and SEQ ID NO:11;

### SEQ ID NO:18 and SEQ ID NO:11;

### SEQ ID NO:19 and SEQ ID NO:11;

### SEQ ID NO:20 and SEQ ID NO:11;

### SEQ ID NO:21 and SEQ ID NO:11;

### SEQ ID NO:22 and SEQ ID NO:11;

### SEQ ID NO:23 and SEQ ID NO:11;

### SEQ ID NO:24 and SEQ ID NO:11;

### SEQ ID NO:25 and SEQ ID NO:11;

### SEQ ID NO:1 and SEQ ID NO:12;

### SEQ ID NO:2 and SEQ ID NO:12;

### SEQ ID NO:3 and SEQ ID NO:12;

### SEQ ID NO:4 and SEQ ID NO:12;

### SEQ ID NO:5 and SEQ ID NO:12;

### SEQ ID NO:6 and SEQ ID NO:12;

### SEQ ID NO:17 and SEQ ID NO:12;

### SEQ ID NO:18 and SEQ ID NO:12;

### SEQ ID NO:19 and SEQ ID NO:12;

### SEQ ID NO:20 and SEQ ID NO:12;

### SEQ ID NO:21 and SEQ ID NO:12;

### SEQ ID NO:22 and SEQ ID NO:12;

### SEQ ID NO:23 and SEQ ID NO:12;

### SEQ ID NO:24 and SEQ ID NO:12;

### SEQ ID NO:25 and SEQ ID NO:12;

### SEQ ID NO:1 and SEQ ID NO:13;

### SEQ ID NO:2 and SEQ ID NO:13;

### SEQ ID NO:3 and SEQ ID NO:13;

### SEQ ID NO:4 and SEQ ID NO:13;

### SEQ ID NO:5 and SEQ ID NO:13;

### SEQ ID NO:6 and SEQ ID NO:13;

### SEQ ID NO:17 and SEQ ID NO:13;

### SEQ ID NO:18 and SEQ ID NO:13;

### SEQ ID NO:19 and SEQ ID NO:13;

### SEQ ID NO:20 and SEQ ID NO:13;

### SEQ ID NO:21 and SEQ ID NO:13;

### SEQ ID NO:22 and SEQ ID NO:13;

### SEQ ID NO:23 and SEQ ID NO:13;

### SEQ ID NO:24 and SEQ ID NO:13;

### SEQ ID NO:25 and SEQ ID NO:13;

### SEQ ID NO:1 and SEQ ID NO:14;

### SEQ ID NO:2 and SEQ ID NO:14;

### SEQ ID NO:3 and SEQ ID NO:14;

### SEQ ID NO:4 and SEQ ID NO:14;

### SEQ ID NO:5 and SEQ ID NO:14;

### SEQ ID NO:6 and SEQ ID NO:14;

### SEQ ID NO:17 and SEQ ID NO:14;

### SEQ ID NO:18 and SEQ ID NO:14;

### SEQ ID NO:19 and SEQ ID NO:14;

### SEQ ID NO:20 and SEQ ID NO:14;

### SEQ ID NO:21 and SEQ ID NO:14;

### SEQ ID NO:22 and SEQ ID NO:14;

### SEQ ID NO:23 and SEQ ID NO:14;

### SEQ ID NO:24 and SEQ ID NO:14;

### SEQ ID NO:25 and SEQ ID NO:14;

### SEQ ID NO:1 and SEQ ID NO:15;

### SEQ ID NO:2 and SEQ ID NO:15;

### SEQ ID NO:3 and SEQ ID NO:15;

### SEQ ID NO:4 and SEQ ID NO:15;

### SEQ ID NO:5 and SEQ ID NO:15;

### SEQ ID NO:6 and SEQ ID NO:15;

### SEQ ID NO:17 and SEQ ID NO:15;

### SEQ ID NO:18 and SEQ ID NO:15;

### SEQ ID NO:19 and SEQ ID NO:15;

### SEQ ID NO:20 and SEQ ID NO:15;

### SEQ ID NO:21 and SEQ ID NO:15;

### SEQ ID NO:22 and SEQ ID NO:15;

### SEQ ID NO:23 and SEQ ID NO:15;

### SEQ ID NO:24 and SEQ ID NO:15;

### SEQ ID NO:25 and SEQ ID NO:15;

### SEQ ID NO:1 and SEQ ID NO:16;

### SEQ ID NO:2 and SEQ ID NO:16;

### SEQ ID NO:3 and SEQ ID NO:16;

### SEQ ID NO:4 and SEQ ID NO:16;

### SEQ ID NO:5 and SEQ ID NO:16;

### SEQ ID NO:6 and SEQ ID NO:16;

### SEQ ID NO:17 and SEQ ID NO:16;

### SEQ ID NO:18 and SEQ ID NO:16;

### SEQ ID NO:19 and SEQ ID NO:16;

### SEQ ID NO:20 and SEQ ID NO:16;

### SEQ ID NO:21 and SEQ ID NO:16;

### SEQ ID NO:22 and SEQ ID NO:16;

### SEQ ID NO:23 and SEQ ID NO:16;

### SEQ ID NO:24 and SEQ ID NO:16; and

### SEQ ID NO:25 and SEQ ID NO:16.

21. The composition according to any one of embodiments 15-20, wherein when used prior to the lautering in a brewing application the total pressure built up is reduced to a value of less than 470 mm WC, such as less than 450 mm WC, such as less than 430 mm WC, such as less than 410 mm WC, such as less than 390 mm WC, such as less than 370 mm WC, such as less than 350 mm WC, such as less than 330 mm WC, such as less than 310 mm WC, such as less than 300 mm WC, such as less than 290 mm WC.

22. The composition according to any one of embodiments 15-21, wherein when used prior to the lautering in a brewing application the total pressure built up is reduced by at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% compared to the use of a negative control without said composition.

23. The composition according to any one of embodiments 15-22, wherein when used in a brewing application prior to the wort separation, the wort filterability as measured by volume wort collected after 5 min of filtration relative to a control without enzymes is increased to above 1.5, such as above 1.6, such as above 1.7, such as above 1.8, such as above 1.9, such as above 2.0, such as above 2.1, such as above 2.2, such as above 2.3, such as above 2.4, such as above 2.5.

24. The composition according to any one of embodiments 15-23, when used in a brewing application prior to the wort separation, the wort filterability as measured by volume wort collected after 5 min of filtration is increased by at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 110, 120, 130, 140, 150, 160, 170, 180, 190, 200, 210, 220, 230, 240, 250, 260, 270, 280, 290, or 300% as compared to the use of a negative control without said composition.

25. The composition according to any one of embodiments 15-24, comprising any one or more further enzyme.

26. The composition according to embodiment 25, wherein said one or more further enzyme is selected from list consisting of a xylanase classified in EC 3.2.1.32, EC 3.2.1.136, or EC 3.2.1.156, a cellulase, a laminarinase, an endo-1,5-α-L-arabinanase, a beta-D-glucoside glucohydrolase, a β-Xylosidase, a cellobiohydrolase, a glucan 1,4-beta-glucosidase, a xyloglucan-specific exo-beta-1,4-glucanase and an α-N-Arabinofuranosidase.

27. Use of an enzyme according to embodiments 1-10, or a preparation according to embodiment 14, or a composition according to any one of embodiments 15-26 in the production of a food, feed, or malt beverage product.

28. Use of an enzyme according to embodiments 1-10, or a preparation according to embodiment 14, or a composition according to any one of embodiments 15-26, in the production of dough or baked products.

29. Use of an enzyme according to embodiments 1-10, or a preparation according to embodiment 14, or a composition according to any one of embodiments 15-26, in the preparation of pulp or paper.

30. Use of an enzyme according to embodiments 1-10, or a preparation according to embodiment 14, or a composition according to any one of embodiments 15-26, for the preparation of cereal components.

31. The use according to embodiment 29, in which the cereal is rye, wheat, or barley.

32. Use of enzyme according to embodiments 1-10, or a preparation according to embodiment 14, or a composition according to any one of embodiments 15-26, in the production of beer or modification of by-products from a brewing process.

33. Use of enzyme according to embodiments 1-10, or a preparation according to embodiment 14, or a composition according to any one of embodiments 15-26, in the production of wine or juice.

34. Use of enzyme according to embodiments 1-10, or a preparation according to embodiment 14, or a composition according to any one of embodiments 15-26, in the production of a first—or second-generation biofuel, such as bioethanol.

35. Method of altering filterability of a starch comprising material, said method comprising the step of treating said starch comprising material with enzyme according to embodiments 1-10, or a preparation according to embodiment 14, or a composition according to any one of embodiments 15-26.

36. Method of reducing pressure built up during lautering in a brewing application, said method comprising the step of treating a brewing mash with enzyme according to embodiments 1-10, or a preparation according to embodiment 14, or a composition according to any one of embodiments 15-26.

37. Method for the production of a food, feed, or beverage product, such as an alcoholic or non-alcoholic beverage, such as a cereal- or malt-based beverage like beer or whiskey, said method comprising the step of treating a starch comprising material with enzyme according to embodiments 1-10, or a preparation according to embodiment 14, or a composition according to any one of embodiments 15-26.

38. Method for the production of a brewing mash, said method comprising the step of treating a starch comprising material with enzyme according to embodiments 1-10, or a preparation according to embodiment 14, or a composition according to any one of embodiments 15-26.

39. Method for the production of a first—or second-generation biofuel, such as bioethanol, said method comprising the step of treating a starch comprising material with an enzyme according to embodiments 1-10, or a preparation according to embodiment 14, or a composition according to any one of embodiments 15-26.

40. Product obtained by the method according to any one of embodiments 38-39.

41. A composition comprising the product according to embodiment 40, such as wherein the product is in a range of 0.1%-99.9%.

### EXAMPLES

### Example 1

### Methods and Results in Relation to Xylanases/Glucanase Filing for Brew Application

The below methods have been used to screen for xylanases and glucanases with application in brewing:

### Methods:

### Water Extractable Arabinoxvlan (WE-AX) Xylanase Method:

Samples, to obtain approx. OD540=0.25-0.30 in this assay and xylose standards (0, 0.125, 0.250, 0.375 and 0.500 mg/ml distilled water) are prepared in distilled water. At time t=0 minutes, 1.75 ml soluble wheat arabinoxylan (0.5% wheat arabinoxylan (PWAXYH, Megazyme, Bray, Ireland)) in 0.1M sodium acetate/acetic acid, pH 5) is placed in a test tube at 50° C. At time t=5 minutes, 250 μl enzyme solution is added to the substrate at 50° C. followed by mixing. Distilled water is used as blank. At time t=15 minutes, 2 ml DNS solution (1% 3,5-Dinitrosalicylic acid (DNS), 1.6% sodium hydroxide, 30% potassium sodium tartrate in distilled water) is added to the enzyme-substrate solution and 2.0 ml standard solution. Samples, blanks and standards added DNS are placed in a boiling water bath (95° C.) for 5 minutes. Hereafter samples, blanks and standards are cooled by placing them in a 25° C. water bath for 20 minutes. The Optical density of all samples are read at OD540 using a spectrophotometer. Based on the dilution of the samples, the amount of sample taking into work and the standards, the xylanases activity of the sample can be calculated.

One Unit of endo-1,4-beta-xylanase WE-AX activity is defined as the amount of enzyme which produces 1 μmole xylose equivalents per minute under the conditions mentioned above (Water extractable arabinoxylan (WE-AX) xylanase method).

### Water Un-Extractable Arabinoxylan (WU-AX) Xylanase Method:

Samples are prepared in distilled water. At time t=0 minutes, 1.75 ml Insoluble wheat (0.5% wheat arabinoxylan (PWAXYI, Megazyme, Bray, Ireland)) in 0.1M sodium acetate/acetic adic, pH 5) is placed in a test tube at 50° C. At time t=5 minutes, 250 μl enzyme solution is added to the substrate at 50° C. followed by mixing. Distilled water is used as blank. At time t=15 minutes, samples and blanks are placed in a boiling water bath (95° C.) for 5 minutes. Hereafter samples and blanks are centrifuged to precipitate residual insoluble substrate. The amount of arabinoxylan brough into solution is determined using the method described by Rouau, X. and Surget, A. (1994), Carbohydrate Polymers, 24, 123-132.

WU-AX endo-1,4-beta-xylanase activity is defined as the amount of pentoses solubilised (μg pentoses) under the conditions described above giving a unit definition of μg pentose/gram of xylanase sample.

### Xylanase Activity Assay

Samples, to obtain approx. OD540=0.25-0.30 in this assay and xylose standards (0, 0.125, 0.250, 0.375 and 0.500 mg/ml distilled water) are prepared in distilled water. At time t=0 minutes, 1.75 ml soluble wheat arabinoxylan (0.5% wheat arabinoxylan (PWAXYH, Megazyme, Bray, Ireland)) in 0.1M sodium acetate/acetic acid, pH 5) is placed in a test tube at 50° C. At time t=5 minutes, 250 μl enzyme solution is added to the substrate at 50° C. followed by mixing. Distilled water is used as blank. At time t=15 minutes, 2 ml DNS solution (1% 3,5-Dinitrosalicylic acid (DNS), 1.6% sodium hydroxide, 30% potassium sodium tartrate in distilled water) is added to the enzyme-substrate solution and 2.0 ml standard solution. Samples, blanks and standards added DNS are placed in a boiling water bath (95° C.) for 5 minutes. Hereafter samples, blanks and standards are cooled by placing them in a 25° C. water bath for 20 minutes. The Optical density of all samples are read at OD540 using a spectrophotometer. Based on the dilution of the samples, the amount of sample taking into work and the standards, the xylanases activity of the sample can be calculated.

One Unit of endo-1,4-beta-xylanase WE-AX activity is defined as the amount of enzyme which produces 1 μmole xylose equivalents per minute under the conditions mentioned above

### Glucanase Activity Assay

Samples, to obtain OD₅₄₀ within the standard curve in this assay and glucose standards (0; 0.125; 0.250; 0.500; and 0.750 mg/ml distilled water) are prepared in distilled water. At time t=0 minutes, 1.75 ml barley beta-glucan (1.5% barley beta-glucan (P-BGBM, Megazyme, Bray, Ireland)) in 1M sodium acetate/acetic adic, pH 5) is placed in a test tube at 50° C. At time t=5 minutes, 250 μl enzyme solution is added to the substrate at 50° C. followed by mixing. Distilled water is used as blank. At time t=15 minutes, 2 ml DNS solution (1% 3,5-Dinitrosalicylic acid (DNS), 1,6% sodium hydroxide, 30% potassium sodium tartrate in distilled water) is added to the enzyme-substrate solution and 2.0 ml standard solution. Samples, blanks and standards added DNS are placed in a boiling water bath (95° C.) for 15 minutes. Hereafter samples, blanks and standards are cooled by placing them in a 25° C. water bath for 20 minutes. The Optical density of all samples are read at OD₅₄₀ using a spectrophotometer. Based on the dilution of the samples, the amount of sample taking into work and the standards, the glucanase activity of the sample can be calculated.

One unit of endo-1,3(4)-β-glucanase activity is defined as the amount of enzyme which produces 1 μmole glucose equivalents per minute under the conditions of the assay (pH 5.0 (or as specified) and 50° C.).

### Lab Scale Brewing Application Method:

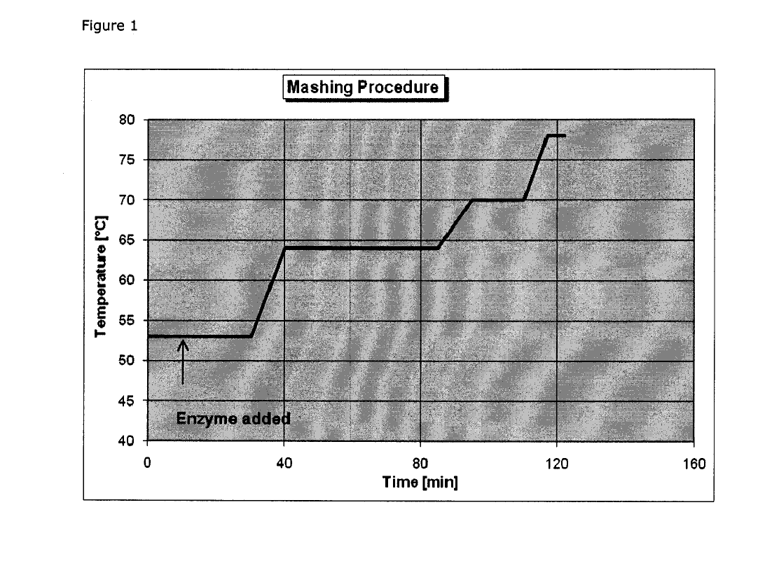

Lab scale brewing application studies were conducted using Pilsner malt:Barley in a 75:25 ratio at a water:grist ratio of 3:1 (150 ml:50 g grist). Initially water was preheated to 53° C. before mashing in and pH adjustment (5.4, 2 M H2SO4). After regaining initial temperature (10 min period) the mashing profile (see FIG. 1) is initiated and enzymes are added. After mashing off wort separation is conducted using a conventional plastic funnel and filter paper (paper filter No 1, 24 cm diameter, Whatman, England). Filtration performance was evaluated as well as several other wort parameters, such as i.e. viscosity, β-glucan and pentosan.

Wort filtration was measured for 30 min after which filtration was terminated. Collected wort was cooled before any further analysis.

### Filtration

Filtration data are presented as volume wort collected after 5, 10, 15 and 30 minutes relative to a blank (brewing without added exogenous enzymes).

### Pilot Scale Brewing

Trials were conducted in a pilot scale brewing facility (2 HL capacity). Wort separation was conducted by lautering and beer filtration by horizontal kiselguhr filtration.

To elucidate filtration optimization by combination of glucanase and xylanase under “challenging” brewing conditions, pilot scale brewing trails were conducted using a mixed grist comprising of 75% malt and 25% barley. Initially, the water:grist ratio was set at 2.8:1 (mash start) increasing to 3.1:1 at the start of lautering. In comparison water:grist ratios around 3.2-3.8 are typical in full scale brew house lautering. Thus the current pilot trial settings of a 3.1:1 water:grist ratio are believed to be in the challenging end of the scale.

Malt and barley was ground dry using a two-roller mill. Both barley and malt was milled twice using a roller distance of ˜0.7 mm.

Mashing-in was conducted aiming at an initial mash temperature of 53° C. After mashing-in small adjustments were conducted such as: mash volume adjustment for water:grist ratio of 2.8:1 and pH adjustment to ˜5.56 (Lactic acid). After fine tuning the mash, enzyme was added and the mashing profile given in FIG. 1 was followed. Saccharification rest at 70° C. was programmed to 15 min, however rest period was extended by 5 min until an iodine test showed that no starch was present. (Ludwig Narziss and Werner Back, Technische Universitaet Muenchen (Fakultaet fuer Brauwesen, Weihenstephan), Abriss der Bierbrauerei. WILEY-VCH Verlags GmbH Weinheim Germany, 2005).

Mashing-off was initiated after a 5 min rest at 78° C. Mash was transferred to the Lauter Tun, which was beforehand prefilled with water to a height just below the “false bottom”. The mash was left to rest for 5 min for settling of filter cake. This was followed by a 15 min recirculation (140 L/h) ensuring filter cake settling and wort clarification. Typically in full scale brewing, filtration will be initiated when a given wort turbidity is obtained, however in the current trials recirculation was kept constant at 15 min enabling comparison of trials. During lautering the following data were collected, including time (min), wort volume collected (L), filtration pressure difference across filter cake (mmWC, mm Water Column), pump capacity (%), wort turbidity (EBC) and mash temperature (° C.).

The pressure build up across the filter cake during filtration is believed to be a factor contributing to setting the standard of the wort lautering performance. Reaching very high pressure differences—e.g. 250 mmWC during first wort collection and e.g. 450 mmWC for the reminder of the lautering—a filter cake racking (also known as deep cut) is induced. Racking is a process where a filter cake collapses or a filtration channel formation is relieved by slowing cutting the filter cake with special designed knives. Following filter cake racking a 6 min wort recirculation (flow rate: 120 l/h) was introduced priming the filter cake for continued filtration. Filter cake racking relieves an otherwise compromised filtration performance which would otherwise also result in poor wort quality. If no pressure induced racking has been introduced by the beginning of the 3rd sparging, automatic rackings were conducted at the beginning of the 3rd and 4th spargings to ensure that no full filtration block would occur just before finishing wort separation.

Lautering was conducted with the settings illustrated in table 1.

TABLE 1
Lautering settings. Volumes collected (L),
filtration flow (L/h) and Sparging volumes (L).

| | Volume | Filtration | Sparging |
|---|---|---|---|
| Wort | collected, L | flow, L/h | volume, L |
| First wort | 0-60 | 130 | |
| 1st sparging | 60-78 | 140 | 18 |
| 2nd sparging | 78-96 | 160 | 18 |
| 3rd sparging | 96-114 | 180 | 18 |

After end lautering, sweet wort was returned to the Mash Tun, heated to boiling and hops were added. Hopping was continued for 80 min and at the end of hopping pH is adjusting to 5.10±0.05. Hops were cleared from the bitter wort by use of whirlpool and following wort was cooled to ˜8° C. For fermentation, a bottom fermenting dried yeast (Saccharomyces cerevisiae) W34/70 from Fermentis was chosen. Yeast was rehydrated for 30 min and pitched at 100 g/HL. Main fermentation was hold for 5-6 days at 10° C., followed by maturation at 15° C. until attenuated and Diacetyl below 80 ppb. Beer was stored for another 2-3 weeks at 1° C. and 0.7 bar before filtering.

Beer was filtered horizontally by use of 1.2 μm PP-candle plates and kieselguhr. Up to 8 plates could be included in the filtration unit, resulting in a total filtration area of ˜0.5 m2. In the current studies 3 plates were included and filtration was conducted at a flow rate of 130 L/h, resulting in a speed of filtration of 6.9 HL/(h·m2). In full scale breweries, speed of filtration is usually set between 5-7 HL/(h·m2). It is thus obvious that the current settings are in the high end—a deliberate choice challenging the beer filtration conditions to verify potential benefits from the choice of using an enzyme in the brewing process. During beer filtration, flow rates (L/h) as well as pressure values (P-in and P-out) were monitored to verify beer filtration performance. Also a number of beer analyses, such as Original Gravity (OG), Apparent Extract (AE), Alcohol By Volume (ABV), Apparent Degree of Fermentation (ADF), Reel Degree of Fermentation (RDF), pH, colour and bitterness were conducted for evaluation of beer quality.

### Results:

### Xylanases:

Xylanases were screened for their activity on soluble substrate and insoluble substrate, their pH and temperature characteristics.

Results are shown in table 2.

TABLE 2
Xylanases screened, their activity on soluble (WE-AX) and insoluble (WU-AX) arabinoxylan
substrate and their biochemical characteristics in regard to temp and pH.

| | | | | | WU-AX/ | Temp opt, | T½ temp, | |
|---|---|---|---|---|---|---|---|---|
| Name | Origin | GH | WE-AX | WU-AX | WE-AX | ° C. | ° C. | pH opt. |
| AfuXyn2 | Aspergillus | 11 | 7798 | 68790526 | 8822 | 65 | 59 | 5.5 |
| | fumingatus | | | | | | | |
| AfuXyn3 | Aspergillus | 11 | 26283 | 99716865 | 3794 | 60 | 62 | 5 |
| | fumingatus | | | | | | | |
| AfuXyn5 | Aspergillus | 11 | 90005 | 714363158 | 7937 | 60 | 50 | 4 |
| | fumingatus | | | | | | | |
| BsuXyn3 | Bacillus subtilis, | 11 | 82 | 1095357 | 13388 | 50 | n.d. | 6 |
| | BS3 | | | | | | | |
| BsuXyn4 | Bacillus subtilis, | 11 | 54 | 1005400 | 18619 | 50 | n.d. | 6 |
| | BS4 #160 | | | | | | | |
| TerXyn1 | Geosmithia | 10 | 1467 | 6208786 | 4232 | 78 | >78 | 3 |
| | emersonii | | | | | | | |
| AtuXyn3 | Aspergillus | 10 | 1220 | 7760982 | 6361 | 65 | 67 | 4.5 |
| | tubigensis | | | | | | | |
| AtuXyn4 | Aspergillus | 11 | 1600 | 12934971 | 8084 | 45 | 58 | 5 |
| | tubigensis | | | | | | | |
| AacXyn2 | Aspergillus | 10 | 777 | 3880491 | 4994 | 70 | 73 | 4 |
| | aculeatus | | | | | | | |
| TreXyn2 | Trichoderma | 11 | 2244 | 16015846 | 7137 | 55 | n.d. | 5 |
| | reesei | | | | | | | |
| TreXyn3 | Trichoderma | 10 | 21487 | 141108772 | 6567 | 60 | 64 | 5.5 |
| | reesei | | | | | | | |
| TreXyn5 | Trichoderma | 11 | 1410 | 8842816 | 6272 | 70 | 68 | 5 |
| | reesei | | | | | | | |
| ApuXyn1 | Aureobasidium | 10 | 3689 | 26810667 | 7.268 | 75 | 76 | 6 |
| | pullulans | | | | | | | |
| TlaXyn1 | Thermomyces | 11 | 15964 | 116918697 | 7.324 | 75 | 72 | 7 |
| | lanuginosus | | | | | | | |
| TauXyn1 | Thermoascus | 10 | 4366 | 19091187 | 4.373 | 75 | >78 | 4.5 |
| | aurantiacus | | | | | | | |
| SthXyn1 | S. | 11 | n.d. | 136405146 | n.d. | 67 | n.d. | 6 |
| | thermoviolaceus | | | | | | | |
| TfuXyn1 | Thermobifida | 11 | n.d. | 30338386 | n.d. | 70 | n.d. | 6.5 |
| | fusca | | | | | | | |
| FoxXyn2* | Fusarium | 10 | n.d. | n.d. | n.d. | 60 | n.d. | 5-6 |
| | oxysporum | | | | | | | |
| FveXyn4* | Fusarium | 10 | n.d. | n.d. | n.d. | 60 | n.d. | 6 |
| | verticilloides | | | | | | | |

*indicates FEED relevant backbones

WE-AX and WU-AX enzyme activities (U) were measured as described in sections “water extractable arabinoxylan (WE-AX) xylanase method” and “water un-extractable arabinoxylan (WU-AX) xylanase method”.

Based on the results from the biochemical screening, xylanases having an appropriate activity ratio on soluble vs. insoluble arabinoxylan were chosen for further testing in application trials. The results are shown in table 3.

TABLE 3
Xylanases screened and the relative extract yield obtained using the
xylanases versus a blank (without xylanases). Finally the xylanases
substrate specificity is illustrated as a ration of their activity on
insoluble vs. soluble arabinoxylan (WU-AX/WE-AX).

| | | Filtration Performance | | | | |
|---|---|---|---|---|---|---|
| | | 5 | 10 | 15 | 30 | WU-AX/ |
| Name | Origin | min | min | min | min | WE-AX |
| Blank | | 1.00 | 1.00 | 1.00 | 1.00 | |
| BsuXyn3 | Bacillus subtilis, | 0.93 | 0.95 | 0.96 | 0.95 | 13388 |
| | BS3 | | | | | |
| BsuXyn4 | Bacillus subtilis, | n.d. | n.d. | n.d. | n.d. | 18619 |
| | BS4 #160 | | | | | |
| TerXyn1 | Geosmithia | 2.19 | 1.92 | 1.70 | 1.44 | 4232 |
| | emersonii | | | | | |
| | (Taleromyces | | | | | |
| | emersonii) | | | | | |
| AtuXyn3 | Aspergillus | 2.06 | 1.75 | 1.59 | 1.37 | 6361 |
| | tubigensis | | | | | |
| AtuXyn4 | Aspergillus | 1.02 | 1.01 | 1.01 | 1.01 | 8084 |
| | tubigensis | | | | | |
| AacXyn2 | Aspergillus | 2.07 | 1.86 | 1.67 | 1.43 | 4994 |
| | aculeatus | | | | | |
| TreXyn3 | Trichoderma | 2.41 | 2.02 | 1.81 | 1.55 | 6567 |
| | reesei | | | | | |
| TreXyn5 | Trichoderma | 2.06 | 1.75 | 1.59 | 1.37 | 6272 |
| | reesei | | | | | |
| Blank | | 1.00 | 1.00 | 1.00 | 1.00 | |
| ApuXyn1 | Aureobasidium | 1.05 | 1.05 | 1.04 | 1.03 | 7.268 |
| | pullulans | | | | | |
| TlaXyn1 | Thermomyces | 1.28 | 1.15 | 1.14 | 1.11 | 7.324 |
| | lanuginosus | | | | | |
| TauXyn1 | Thermoascus | 1.22 | 1.18 | 1.18 | 1.14 | 4.373 |
| | aurantiacus | | | | | |
| SthXyn1 | S. | 1.30 | 1.15 | 1.13 | 1.10 | n.d. |
| | thermoviolaceus | | | | | |
| TfuXyn1 | Thermobifida | 1.20 | 1.10 | 1.06 | 1.06 | n.d. |
| | fusca | | | | | |
| FoxXyn2* | Fusarium | 1.20 | 1.08 | 1.09 | 1.08 | n.d. |
| | oxysporum | | | | | |
| FveXyn4* | Fusarium | 1.20 | 1.07 | 1.06 | 1.04 | n.d. |
| | verticilloides | | | | | |

*indicates FEED relevant backbones

Filtration performance was measured as described earlier (“filtration”), and is presented as volume filtrate at the different time points relative to the negative control (blank).

WE-AX and WU-AX enzyme activities (U) were measured as described in sections “water extractable arabinoxylan (WE-AX) xylanase method” and “water un-extractable arabinoxylan (WU-AX) xylanase method”.

### Glucanases:

Glucanases were screened for their activity and temperature characteristics, and the results are shown in table 4.

TABLE 4
Glucanases screened, their activity and their biochemical characteristics in
regard to temperature

| | | | | T½ | T½ | |
|---|---|---|---|---|---|---|
| | | | Temp | temp_buffer, | temp_wort, | |
| Name | Origin | U/ml | opt, ° C. | ° C. | ° C. | pH opt. |
| TerGlu1 | Talaromyces | 7338 | 70 | 78 | 78 | 3 |
| | emersonii/ | | | | | |
| | Geosmithia | | | | | |
| | emersonii | | | | | |
| BsuGluS | Bacillus subtilis | 208 | 55-65 | 60 | 68 | 5-6 |
| BsuGlu103FULL | Bacillus subtilis | 391 | 50-60 | 53 | 58 | 5-6 |
| TreGlu2 | Trichoderma reesei | 13 | 40-50 | 70 | 74 | 4.5-6 |
| TreGlu3 | Trichoderma reesei | 9215 | 40-51 | 58 | 62 | 4.5-6 |
| TreGlu4 | Trichoderma reesei | n.d. | 40-52 | 62 | 62 | 4.5-6 |
| TreGlu6 | Trichoderma reesei | n.d. | 40-53 | 62 | 64 | 4.5-6 |
| TreGlu7 | Trichoderma reesei | n.d. | 40-54 | 62 | 62 | 4.5-6 |
| TreGlu8 | Trichoderma reesei | n.d. | 40-55 | 61 | 63 | 4.5-6 |
| BsuGluC CBD | Bacillus subtilis | 10 | 50-60 | 60 | 67 | 5-6 |

n.d. = Not determined

Glucanase activity/units was/were determined as described in the glucanase activity assay as described above.

Based on the results from the biochemical screening, glucanases having suitable characteristics were chosen for further testing in application trials. The results are shown in table 5.

TABLE 5
Name and origin of glucanases screened and
the relative extract yield obtained using
the glucanases versus a blank (without enzyme).

| | | Filtration performance | | | |
|---|---|---|---|---|---|
| | | 5 | 10 | 15 | 30 |
| Name | Origin | min | min | min | min |
| Blank | Neg control | 1.00 | 1.00 | 1.00 | 1.00 |
| TerGlul | Geosmithia emersonii | 1.36 | 1.43 | 1.46 | 1.36 |
| BsuGluS | Bacillus subtilis | 1.48 | 1.49 | 1.48 | 1.35 |
| BsuGlu103FULL | Bacillus subtilis | 1.29 | 1.28 | 1.30 | 1.22 |
| TreGlu2 | Trichoderma reesei | 1.15 | 1.18 | 1.20 | 1.15 |
| TreGlu3 | Trichoderma reesei | 1.29 | 1.32 | 1.30 | 1.22 |
| TreGlu4 | Trichoderma reesei | 1.11 | 1.11 | 1.11 | 1.09 |
| TreGlu6 | Trichoderma reesei | 1.13 | 1.15 | 1.13 | 1.10 |
| TreGlu7 | Trichoderma reesei | 1.06 | n.d. | 1.01 | 1.02 |
| TreGlu8 | Trichoderma reesei | 1.12 | 1.11 | 1.13 | 1.09 |
| BsuGluC CBD | Bacillus subtilis | 1.33 | 1.37 | 1.37 | 1.32 |

Filtration performance was measured as described earlier (“filtration”), and is presented as volume filtrate at the different time points relative to the negative control (blank).

Based on the individual screening of xylanases and glucanases, combinatorial experiments were conducted, and results are illustrated in table 6.

TABLE 6
Brewing application results from combinatorial experiments of
xylanases and glucanases versus a blank and versus UltraFlo ®Max.
250 Fungal Xylanase Units FXU-S/g; 700 Cellulase Units EGU/g
(Novozymes, Denmark) results are illustrated as relative extract yield obtained.

| | | Filtration performance | | | |
|---|---|---|---|---|---|
| Name | Origin | 5 min | 10 min | 15 min | 30 min |
| Control | | 1.00 | 1.00 | 1.00 | 1.00 |
| UFmax 0.1 | A. aculeatus | 2.29 | 2.13 | 2.00 | 1.77 |
| BsuGluS/TauXyn1 | B. sub/T. aurantiacus | 1.70 | 1.69 | 1.60 | 1.47 |
| BsuGluS/AtuXyn3 | B. sub/A. tubingensis | 2.57 | 2.14 | 1.96 | 1.75 |

(Origin of UltraFlo ® Max may include other microorganisms than A. aculeatus, such as described in WO05059084)

Filtration performance was measured as described earlier (“filtration”), and is presented as volume filtrate at the different time points relative to the negative control (blank).

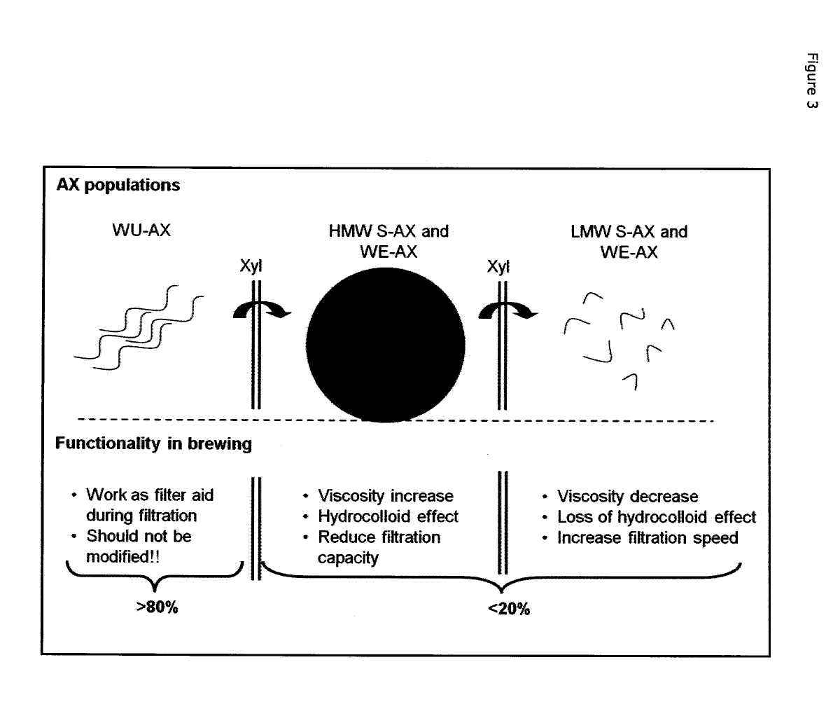

Suitable combinations were further tested in a 2HL pilot scale facility for verification, and results are shown in table 7 and FIG. 3.

TABLE 7
Pilot scale Brewing application results from verification of the glucanase
and xylanases screening. The B. sub glucanase S combined with the A. tub
xylanases were tested against a blank and UltraFlo ®Max. Data collected
was the average flow (L/h), the total pressure build up over the lautering
(mm WC) and the max pressure recorded during the lautering (mm WC).

| | | Total | |
|---|---|---|---|
| | Avg Flow | pressure build up | Max pressure |
| ID | (L/h) | (mm WC) | (mm WC) |
| Blank | 148 | 556 | 356 |
| UltraFlo max | 149 | 478 | 280 |
| BsuGluS/AtuXyn3 | 147 | 263 | 163 |

### Example 2

In this example it was attempted to show that xylanases for brewing applications may have a very high selectivity for High Molecular Weight Soluble-arabinoxylan (HMWS-AX) and water extractable arabinoxylan (WE-AX). It is believed that hereby only limited amounts of arabinoxylan need to be solubilised. Consequently, the related off flavour potential is highly reduced.

A significantly reduced viscosity is facilitating mash and beer separation. Desired xylanase characteristics for brewing applications may include one or more of the following aspects of table 8:

TABLE 8

Screening criterias for xylanase selection
Enzyme substrate specificity
WE-AX/WU-AX ratio has an impact on viscosity
Enzyme substrate selectivity
How close to branch points the enzyme will cut has an
impact on the functionality
Enzyme thermostability
Continuous solubilisation of AX during mashing-
thermostability is a key feature
Enzyme pH optimum (pH 5.4-5.6)
Enzyme inhibition (e.g. known key factor for xylanases)

TABLE 9
Xylanases-biochem characteristics
Inhibition by endogenous cereal xylanase
inhibitors occur in both xylanase GH's

| Xylanase GH | GH10 | GH11 |
|---|---|---|
| Mw | +30 kDa | 20 kDa |
| Substrate | Hydrolyse close to | Need more |
| specificity | Arabinose | unsubstituted |
| | substitutions | Xylose to |
| | | hydrolyse AX |
| Substrate | WE-AX/WU-AX | WE-AX/WU-AX |
| selectivity | typically >1 | typically <1 |
| SBD | Often separate | No classical SBD, |
| | SBD | but secondary BD |
| | | on surface |
| Technological | Viscosity reducers | Solubilizer/ |
| effect | | viscosity reducers |

Water-Unsoluble ArabinoXylan (WU-AX) in cereals as shown in FIG. 3 is linked to filter cake stability in the brew house.

The concentration of ferulic acid (FA) in cereals very much depends on the tissue. The highest concentration is found in the pericarp material, whereas the concentration in the endosperm is much lower. Different concentrations are reported. A concentration of 2700 μg/g insoluble fiber, 185 μg/g soluble fiber is likely (Bunzel et al. 2001, Journal of Sc. of food and agriculture, vol. 81, p. 653-60).

To put this into perspective it means that FA is only found for every 200th xylose molecules in arabinoxylan in insoluble fiber (WU-AX) and for every 2500 xylose in soluble fiber (WE-AX).

It is a well-known fact that xylanases may lead to off-flavor formation in beer such as free ferulic acid and 4-VG.

### Methods:

Based on the criteria mentioned in table 8+9 more than 15 xylanases from DuPont Industrial Biosciences were found as potential candidates. The xylanases were screened in laboratory mashing application applying up to 30% barley in combination with malt. Among others, mash separation speed, pentosan/arabinoxylan level and wort viscosities were monitored. Top candidates where tested at several pilot brewery plant studies to test our hypothesis and link xylanase characteristics to functionality in brewing. The optimal dosage of the selected xylanase candidate was tested in combination with a β-glucanase.

### Results and Discussion:

TABLE 10

| | | Ref | | | |
|---|---|---|---|---|---|
| Sample ID | Control | (X + B). | X1 | X2 | X3 |
| Dyn. Viscosity (12° Plato) | 1.798 | 1.670 | 1.801 | 1.746 | 1.794 |
| mPa · s | | | | | |
| Extract (° Plato) | 15.1 | 15.7 | 15.6 | 15.2 | 15.1 |
| Total pentosan (mg/l) | 1610 | 1910 | 2440 | 2020 | 1710 |

Pilot plant brews where enzyme dosage is the only variable. Applying a WU-AX selective xylanase (X1) results in filter bed collapse. WE-AX selective xylanase candidates (reference, X2, X3) results in low pressure buildup. The reference is a blend of xylanase+beta-glucanase.

TABLE 11
Wort analyses-pilot plant studies

| Sample ID | Ref. | X + B | Xh + B |
|---|---|---|---|
| Extract (° Plato) | 15.70 | 16.00 | 15.95 |
| Betaglucan in wort (mg/l) | 44 | 35 | 25 |
| Dyn. Viscosity at 12° Plato | 1.65 | 1.68 | 1.68 |
| (mPa · s) | | | |
| Total pentosan (mg/l) | 3540 | 2970 | 3010 |

TABLE 12
Strecker Aldehyd analysis of aged beer

| Aging markers | | | | |
|---|---|---|---|---|
| (forced aged beer) | Unit | Ref. | X + B | Xh + B |
| 2-Me-Pr | ppb | 25 | 24 | 22 |
| 2-Me-Bu | ppb | 3 | 2 | 3 |
| 3-Me-Bu | ppb | 9 | 7 | 8 |
| Furfural | ppb | 113 | 85 | 93 |
| Methional | ppb | 6 | 5 | 6 |
| PheAcal | ppb | 10 | 9 | 10 |
| T2N | ppb | 0.022 | 0.022 | 0.022 |

Optimized blends of a WE-AX selective xylanase applied at a medium (X) and a high dosage (Xh) in combination with β-glucanase (B) on 20% barley/80% malt. The results indicate a good mash and beer separation performance with a low risk of off-flavor formation and filter bed collapse.

### Conclusion

The study has proven the importance of applying xylanases for brewing which are highly selective for the WE-AX during mashing. The following benefits are achieved:
Good mash separation and beer filtration performance
Minimized risk of filter bed collapse at lautering
Reduced potential for off-flavor formation related to arabinoxylan breakdown
Tolerance towards xylanase overdose

Xylanases can often be applied with a high beneficial effect in combination with beta-glucanases for separation control.

### Example 3

### Evaluation of X3/BgIS (Also Referred to as AtuXyn3/BsuGluS) Combinations in 2 HL Pilot Brewing Trials

### Material and Methods:

### Experiments:Enzymes:

AtuXyn3 (X3)/BsuGluS (Bgls) (a): Combination of BgIS (Bacillus glucanase) and X3 (Aspergillus xylanase; BgLS: 0.50 mg protein/kg grist and X3: 1.50 mg protein/kg grist).

AtuXyn3 (X3)/BsuGluS (Bgls) (b): As AtuXyn3 (X3)/BsuGluS (Bgls) (a), but with 20% increase X3 dose to test robustness.

Reference: Benchmark enzyme product (Ultraflo® Max) dosed at 0.20 kg/T grist.

### Raw Material:

Adjunct material: barley 22% w/w.
Malt: Pilsner malt Chiraz 42.6% w/w, Pilsner malt Quench DMG 35.4% weight pr. weight (ww).

All material used for acid adjustment of pH, Calcium, Zink and bitterness levels are food grade and considered as standard brewing materials.

The recipe for the brew was aiming at a beer style as an international lager beer.

### Milling:

Künzel 2 roller pilot mill. The milled material was passing the rollers twice simulating a 4 roller mill.
Malt grist: the mill was running at 1.5 mm at the first pass and 0.7 mm at the second pass of the rollers.
Barley grist: the mill was running at 1.5 mm at the first pass and 0.4 mm at the second pass of the rollers.

### Brewhouse 2 HL:

All brews were based on HGB (High Gravity Brewing) infusion mashing and standard lautering of 190 L wort aiming at 16° Plato. During lautering which was performed at fixed flow, the differential pressure was recorded (used as parameter for evaluating lautering performance). All brew materials were milled ahead of time (24 h) and kept in closed buckets prior to water contact. All material was dumped in the mash kettle within the first 3 minutes after start of mashing. Calcium and pH adjustment were done prior to enzyme addition. pH (20° C.) was rechecked at the 52° C. break. Iodine normality was confirmed after 10 minutes at 72° C. Lautering was performed at 78° C.

Lautering performance was evaluated on fixed flow at 90 I/H during first wort collection. Flow was increased to 110 L/hour and 130 L/hour during sparging and weak wort collection. Chemical analysis was performed on cold wort.

### Wort Boiling:

Boiling was performed using an external boiler with 4-5% evaporation. Hop extracts were added from the beginning of the wort boiling aiming at 20 BU in the final beer.

### Fermentation 50 L:

All fermentations were performed in 50 L cylindriconical tanks. Fermentation was made according to standard operation procedures. Pitching was done with 15×10⁶ live yeast cells/ml. Yeast counts and viability was calculated using a Nucleo counter.

### Beer Processing:

Plate and frame filter operated at constant pressure. Flow evaluation was done by weight.

Data was collected from 1 and 3 filter plates.

### Debrewing:

All beers were de-brewed to 5.0% ABV (Alcohol By Volume), considered as international lager beer standard.

### Bottling:

CO₂ was adjusted to 5.0 g/L. All beer samples were bottled in 33 cl standard bottles on a McLennon automatic filling machine using single evacuation.

### Beer Analysis:

Fresh beers were analysed using GC-MS

Chemical aging profile was determined using GC-MS.

Results and observations: Mashing.

Mashing was performed with the following condition:

52° C. for 10 minutes simulating a 15 −20 minutes mashing using a running mill.
65° C. for 40 minutes.
72° C. for 30 minutes.
78° C. for 10 minutes.

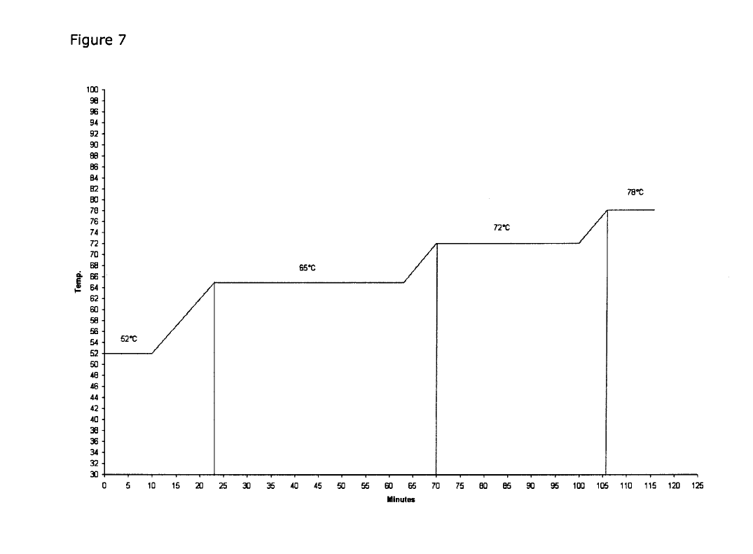

All ramping steps were executed at 1° C. A graphic representation is given in FIG. 7.

All trials were made with this mashing regime aiming at a 16° Plato brew. There were no remarks to this process step.

### Results and Observations: Lautering.

The lautering was performed in the 2 hl brewery with a load of 150 kg/m². This is representative for a standard brew house operation. Control of the lautering process was made as a fixed flow at an average of 100 litre/hour. Initial flow rate is 90 litre/hour, increasing to 130 litre/hour during weak wort collection. Differential pressure and in-line measurement of haze was recorded for the four brews. Total lautering and wort collection was done over approximately 2 hours.

Trial X3/BgIS (b) and X3/BgIS (a) are suggested to be the trials that had the best lautering performance followed by trial X3/BgIS (a) and trial UF max with the worst performance.

TABLE 13
Data collected during lautering of the mash from the four trials.

| | UF max | X3/BglS (a) | X3/BglS (b) | X3/BglS (a) |
|---|---|---|---|---|
| Lauter tun load (kg/m3) | 153 | 153 | 153 | 153 |
| Lauter tun time (min) | 154 | 164 | 170 | 154 |
| Diff. Pressure (cm) | 40 | 30 | 30 | 30 |
| Racking (# deep cuts) | 1 | 1 | 1 | 1 |
| Haze (EBC) | 10 | 15 | 10 | 10 |
| First wort pressure build up (cm/h) | 40 | 33 | 31 | 30 |
| Time to first deep cut (min) | 45 | 60 | 120 | 115 |

“Diff. Pressure” and “First wort pressure build up” in the table was measured as cmWC(cm Water column) and not as (cm) and (cm/h) respectively.

Results and observations: Wort analysis after boiling.

Analysis of the cold wort shows similar results. The beta-glucan analysis indicates a slight difference between the samples.

TABLE 14
Chemical analysis of the cold wort.

| Wort | UF max | X3/BglS (a) | X3/BglS (b) | X3/BglS (a) |
|---|---|---|---|---|
| Extract (% plato) | 16.09 | 16.05 | 15.99 | 16.1 |
| Color (EBC) | 9.7 | 9.3 | 9.3 | 9.3 |
| pH | 5 | 5.2 | 5.2 | 5.2 |
| Iodine (Y/N) | N | N | N | N |
| Bitterness | 52 | 51 | 46 | 50 |
| (BU, EBC) | | | | |

TABLE 15
Analytical data on cold wort.

| | UF max | X3/BglS (a) | X3/BglS (b) | X3/BglS (a) |
|---|---|---|---|---|
| Beta-glucan in wort (mg/L) | 49 | 40 | 25 | 30 |
| Dyn. Viscosity at 12° C. (mPa · s) | 1,888 | 1,685 | 1,679 | 1,686 |
| Pentosan (mg/l) | 3365 | 2975 | 3014 | 2964 |
| Ferulic acid (ug/ml) | 4.3 | 3.9 | 3.8 | 3.9 |
| 4-VG (ug/ml) | <0.49 | <0.49 | <0.49 | <0.49 |

(12° C. is 12° Plato);
(% Plato may be used interchangeably with ° Plato)

Results and observations: Fermentation.

Analysis of the green beer is given in table 16.

TABLE 16
Green beer analysis.

| Green beer | UF max | X3/BglS (a) | X3/BglS (b) | X3/BglS (a) |
|---|---|---|---|---|
| Alcohol (% vol) | 6.79 | 6.79 | 6.7 | 6.86 |
| Real extract (% P) | 6.28 | 6 | 6 | 6 |
| RDF (%) | 63.5 | 63.7 | 63.5 | 64.4 |
| Original extract | 16.29 | 16.25 | 16.09 | 16.24 |
| (% P) | | | | |
| Color (EBC) | 8.3 | 8.5 | — | — |
| pH | 4.4 | 4.4 | 4.4 | 4.4 |
| SO2 (ppm) | 7 | 9 | 11 | 10 |
| Bitterness | 29 | 28 | 27 | 27 |
| (BU, EBC) | | | | |

The green beer analysis shows a high degree of similarity between the trials. All trials have relative low RDF, but this is normally seen with the inclusion of 22% barleycalculated on the basis of weight per weight (ww).

Results and observations: Beer filtration.

Beer samples were filtered using a plate and frame filter using a fixed pressure. Two kegs of approximately 15 kg were filtered and the individual keg filtration data are presented in table 17. The first keg was filtered using 1 filter sheet and the second keg was filtered using 3 filter sheets. The differential pressure was always 0.5 bar. The filter plates are KD7 (20 cm×20 cm) from Begerow.

The overall picture of the filtrations curves from either 1 or 3 filter plate filtrations is the same. We believe that the 1 filter plate record may be too sensitive to show the real ratio difference.

TABLE 17
Keg filtration data from the four trials

| Filtration | UF max | X3/BglS (a) | X3/BglS (b) | X3/BglS (a) |
|---|---|---|---|---|
| Filtration speed- | 4.8 | 5.6 | 9.9 | 11.8 |
| 1 filter sheet (L/h) | | | | |
| Filtration speed- | 77.2 | 59.6 | 70.6 | 105.4 |
| 3 filter sheet (L/h) | | | | |

Results and observations: Final beer analysis.

Trial beers were analysed according to standard operation procedures (EBC) and presented in table 18.

TABLE 18
Final beer analysis.

| Finished beer | UF max | X3/BglS (a) | X3/BglS (b) | X3/BglS (a) |
|---|---|---|---|---|
| alcohol (%) | 4.82 | 4.89 | 5.01 | 4.92 |
| Real extract (% P) | 4.5 | 4.6 | 4.6 | 4.4 |
| RDF (%) | 63.2 | 63.4 | 63.8 | 64.3 |
| Original extract (% P) | 11.85 | 11.99 | 12.19 | 11.89 |
| Color (EBC) | 4.8 | 4.9 | 5 | 5 |
| pH | 4.4 | 4.4 | 4.4 | 4.4 |
| SO2 (ppm) | 13 | 13 | 10 | 6 |
| Bitterness (BU, EBC) | 22 | 22 | 20 | 18 |
| Haze (EBC) | 0.43 | 0.4 | 0.38 | 0.4 |
| Total haze-5 d-60 dg C. (EBC) | 8.6 | 12.9 | 7.3 | 6.2 |
| CO2 (g/L) | 4.9 | 5.3 | 5.1 | 5.2 |
| Diacetyl (ppb) | 12 | 11 | 8 | 10 |
| Head retention (S) | 107 | 111 | 119 | 108 |
| Foam volume (ml) | 452 | 476 | 460 | 470 |

Results and observations: Strecker aldehydes and “age markers” in final beer.

Analysis was performed both on fresh and aged beer. Strecker aldehydes and the “age and heat markers” (2-Me-Pr (2-methyl Propanal), 2-Me-Bu (2-methyl Butanal), 3-Me-Bu (3-methyl Butanal), Furfural, Methional, PheAcal (phenyl Acetaldehyde) and T2N (trans-2-nonenal)) were analysed by GC-MS on both fresh and aged beer. The data from analysis of fresh beer is presented in table 19.

TABLE 19
Strecker aldehyde analysis of fresh beer. Markers for heat
and aging (Furfural and trans-2-Nonenal) are used as sample control.

| Aging markers | | | | |
|---|---|---|---|---|
| (fresh beer) | UF max | X3/BglS (a) | X3/BglS (b) | X3/BglS (a) |
| 2-ME-Pr (ppb) | 5 | 5 | 5 | 6 |
| 2-ME-Bu (ppb) | 2 | 2 | 2 | 2 |
| 3-ME-Bu (ppb) | 6 | 6 | 6 | 6 |
| Furfural (ppb) | 10 | 11 | 11 | 10 |
| Methional (ppb) | 4 | 4 | 4 | 4 |
| PheAcal (ppb) | 6 | 6 | 6 | 7 |
| T2N (ppb) | 0.0011 | 0.005 | 0.004 | 0.006 |

The trial beers were incubated at 37° C. for 2 weeks prior to the Strecker aldehyde analysis. The data for aged beer samples are presented in table 20.

TABLE 20
Strecker aldehyde analysis of aged beer. Markers for heat
and aging (furfural and trans-2-Nonenal) is used as a sample control.

| Aging markers | | | | |
|---|---|---|---|---|
| (forced aged beer) | UF max | X3/BglS (a) | X3/BglS (b) | X3/BglS (a) |
| 2-ME-Pr (ppb) | 25 | 23 | 22 | 26 |
| 2-ME-Bu (ppb) | 3 | 3 | 3 | 2 |
| 3-ME-Bu (ppb) | 9 | 7 | 7 | 8 |
| Furfural (ppb) | 111 | 92 | 93 | 78 |
| Methional (ppb) | 6 | 5 | 6 | 6 |
| PheAcal (ppb) | 10 | 9 | 9 | 10 |
| T2N (ppb) | 0.017 | 0.022 | 0.022 | 0.022 |

The data presented in table 20 show an expected increase in Strecker aldehyde level. The increase in furfural and trans-2-Nonenal reach an expected level.

### Conclusion:

Based on the pilot scale experiments, we can conclude that the ratios of the BgIS and X3 tested in this Experiment performs as good or even better than the reference UltraFlo Max in pilot scale brewing.

The results are surprising, seen in the light of the challenging raw material used, 22% barley inclusion in combination with the 300 mg/l β-glucan containing malt. The performance is not only seen in the mash separation results, also in the beer filtration. Due to the low solubilisation of cell wall material when using the BREW2 (pentosan data), a lower degree of cell wall material that might cause quality issues in relation to off-taste and stability, can be recorded.

Finally it can be concluded that a 20% increase in the dose of the xylanase component in X3/Bgls (b) appears not to have any impact on any of the evaluated parameters, indicating that X3/Bgls (a) is a robust enzyme combination.

### Sequences:

AtuXyn3, Aspergillus tubigensis, 302 aa
(SEQ ID NO: 1)
QASVSIDTKFKAHGKKYLGNIGDQYTLTKNSKTPAIIKADFGALTPENSM
KWDATEPSRGQFSFSGSDYLVNFAQSNNKLIRGHTLVWHSQLPSWVQAIT
DKNTLIEVMKNHITTVMQHYKGKIYAWDVVNEIFNEDGSLRDSVFYQVIG
EDYVRIAFETARAADPNAKLYINDYNLDSASYPKLTGMVSHVKKWIEAGI
PIDGIGSQTHLSAGGGAGISGALNALAGAGTKEIAVTELDIAGASSTDYV
EVVEACLDQPKCIGITVWGVADPDSWRSSSTPLLFDSNYNPKPAYTAIAN
AL
TerXyn1, Geosmithia emersonii (Taleromyces
emersonii)
(SEQ ID NO: 2)
AGLNTAAKAIGLKYFGTATDNPELSDTAYETQLNNTQDFGQLTPANSMKW
DATEPEQNVFTFSAGDQIANLAKANGQMLRCHNLVWYNQLPSWVTSGSWT
NETLLAAMKNHITNVVTHYKGQCYAWDVVNEALNDDGTYRSNVFYQYIGE
AYIPIAFATAAAADPNAKLYYNDYNIEYPGAKATAAQNLVKLVQSYGARI
DGVGLQSHFIVGETPSTSSQQQNMAAFTALGVEVAITELDIRMQLPETEA
LLTQQATDYQSTVQACANTKGCVGITVWDWTDKYSWVPSTFSGYGDACPW
DANYQKKPAYEGILTGLGQTVTSTTYIISPTTSVGTGTTTSSGGSGGTTG
VAQHWEQCGGLGWTGPTVCASGYTCTVINEYYSQCL
AtuXyn4, Aspergillus tubigensis
(SEQ ID NO: 3)
EPIEPRQASVSIDTKFKAHGKKYLGNIGDQYTLTKNSKTPAIIKADFGAL
TPENSMKWDATEPSRGQFSFSGSDYLVNFAQSNNKLIRGHTLVWHSQLPS
WVQSITDKNTLIEVMKNHITTVMQHYKGKIYAWDVVNEIFNEDGSLRDSV
FYKVIGEDYVRIAFETARAADPNAKLYINDYNLDSASYPKLTGMVSHVKK
WIAAGIPIDGIGSQTHLSAGGGAGISGALNALAGAGTKEIAVTELDIAGA
SSTDYVEVVEACLNQPKCIGITVWGVADPDSWRSSSTPLLFDSNYNPKPA
YTAIANAL
AacXyn2, Aspergillus aculeatus
(SEQ ID NO: 4)
MVGLLSITAALAATVLPNIVSAVGLDQAAVAKGLQYFGTATDNPELTDIP
YVTQLNNTADFGQITPGNSMKWDATEPSQGTFTFTKGDVIADLAEGNGQY
LRCHTLVWYNQLPSWVTSGTWTNATLTAALKNHITNVVSHYKGKCLHWDV
VNEALNDDGTYRTNIFYTTIGEAYIPIAFAAAAAADPDAKLFYNDYNLEY
GGAKAASARAIVQLVKNAGAKIDGVGLQAHFSVGTVPSTSSLVSVLQSFT
ALGVEVAYTEADVRILLPTTATTLAQQSSDFQALVQSCVQTTGCVGFTIW
DWTDKYSWVPSTFSGYGAALPWDENLVKKPAYNGLLAGMGVTVTTTTTTT
TATATGKTTTTTTGATSTGTTAAHWGQCGGLNWSGPTACATGYTCTYVND
YYSQCL
TreXyn3, Trichoderma reesei
(SEQ ID NO: 5)
MKANVILCLLAPLVAALPTETIHLDPELAALRANLTERTADLWDRQASQS
IDQLIKRKGKLYFGTATDRGLLQREKNAAIIQADLGQVTPENSMKWQSLE
NNQGQLNWGDADYLVNFAQQNGKSIRGHTLIWHSQLPAWVNNINNADTLR
QVIRTHVSTVVGRYKGKIRAWDVVNEIFNEDGTLRSSVFSRLLGEEFVSI
AFRAARDADPSARLYINDYNLDRANYGKVNGLKTYVSKWISQGVPIDGIG
SQSHLSGGGGSGTLGALQQLATVPVTELAITELDIQGAPTTDYTQVVQAC
LSVSKCVGITVWGISDKDSWRASTNPLLFDANFNPKPAYNSIVGILQ
TreXyn5, Trichoderma reesei
(SEQ ID NO: 6)
QCIQPGTGYNNGYFYSYWNDGHGGVTYCNGPGGQFSVNWSNSGNFVGGKG
WQPGTKNRVINFSGSYNPNGNSYLSVYGWSRNPLIEYYIVENFGTYNPST
GATKLGEVTSDGSVYDIYRTQRVNQPSIIGTATFYQYWSVRRNHRSSGSV
NTANHFNAWAQQGLTLGTMDYQIVAVEGYFSSGSASITVSD
BsuGluS, Bacillus subtilis, 214 aa
(SEQ ID NO: 7)
QTGGSFFDPFNGYNSGFWQKADGYSNGNMFNCTWRANNVSMTSLGEMRLA
LTSPAYNKFDCGENRSVQTYGYGLYEVRMKPAKNTGIVSSFFTYTGPTDG
TPWDEIDIEFLGKDTTKVQFNYYTNGAGNHEKIVDLGFDAANAYHTYAFD
WQPNSIKWYVDGQLKHTATNQIPTTPGKIMMNLWNGTGVDEWLGSYNGVN
PLYAHYDWVRYTKK
TerGlu1, Geosmithia emersonii (Taleromyces
emersonii)
(SEQ ID NO: 8)
APVKEKGIKKRASPFQWFGSNESGAEFGNNNIPGVEGTDYTFPNTSAIQI
LIDQGMNIFRVPFLMERMVPNQMTGPVDSAYFQGYSQVINYITSHGASAV
IDPHNFGRYYNNIISSPSDFQTFWHTIASNFADNDNVIFDTNNEYHDMDE
SLVVQLNQAAIDGIRAAGATSQYIFVEGNSWTGAWTWTQVNDAMANLTDP
QNKIVYEMHQYLDSDGSGTSDQCVNSTIGQDRVESATAWLKQNGKKAILG
EYAGGANSVCETAVTGMLDYLANNTDVWTGAIWWAAGPWWGDYIFSMEPP
SGIAYEQVLPLLQPYL
BsuGlu103FULL, Bacillus subtilis
(SEQ ID NO: 9)
DDYSVVEEHGQLSISNGELVNERGEQVQLKGMSSHGLQWYGQFVNYESMK
WLRDDWGITVFRAAMYTSSGGYIDDPSVKEKVKETVEAAIDLGIYVIIDW
HILSDNDPNIYKEEAKDFFDEMSELYGDYPNVIYEIANEPNGSDVTWDNQ
IKPYAEEVIPVIRDNDPNNIVIVGTGTWSQDVHHAADNQLADPNVMYAFH
FYAGTHGQNLRDQVDYALDQGAAIFVSEWGTSAATGDGGVFLDEAQVWID
FMDERNLSWANWSLTHKDESSAALMPGANPTGGWTEAELSPSGTFVREKI
RESASIPPSDPTPPSDPGEPDPGEPDPTPPSDPGEYPAWDSNQIYTNEIV
YHNGQLWQAKWWTQNQEPGDPYGPWEPLKSDPDSGEPDPTPPSDPGEYPA
WDSNQIYTNEIVYHNGQLWQAKWWTQNQEPGDPYGPWEPLN
TreGlu2, Trichoderma reesei
(SEQ ID NO: 10)
QQTVWGQCGGIGWSGPTNCAPGSACSTLNPYYAQCIPGATTITTSTRPPS
GPTTTTRATSTSSSTPPTSSGVRFAGVNIAGFDFGCTTDGTCVTSKVYPP
LKNFTGSNNYPDGIGQMQHFVNDDGMTIFRLPVGWQYLVNNNLGGNLDST
SISKYDQLVQGCLSLGAYCIVDIHNYARWNGGIIGQGGPTNAQFTSLWSQ
LASKYASQSRVWFGIMNEPHDVNINTWAATVQEVVTAIRNAGATSQFISL
PGNDWQSAGAFISDGSAAALSQVTNPDGSTTNLIFDVHKYLDSDNSGTHA
ECTTNNIDGAFSPLATWLRQNNRQAILTETGGGNVQSCIQDMCQQIQYLN
QNSDVYLGYVGWGAGSFDSTYVLTETPTGSGNSWTDTSLVSSCLARK
TreGlu3, Trichoderma reesei
(SEQ ID NO: 11)
QTSCDQWATFTGNGYTVSNNLWGASAGSGFGCVTAVSLSGGASWHADWQW
SGGQNNVKSYQNSQIAIPQKRTVNSISSMPTTASWSYSGSNIRANVAYDL
FTAANPNHVTYSGDYELMIWLGKYGDIGPIGSSQGTVNVGGQSWTLYYGY
NGAMQVYSFVAQTNTTNYSGDVKNFFNYLRDNKGYNAAGQYVLSYQFGTE
PFTGSGTLNVASWTASIN
TreGlu4, Trichoderma reesei
(SEQ ID NO: 12)
HGHINDIVINGVWYQAYDPTTFPYESNPPIVVGWTAADLDNGFVSPDAYQ
NPDIICHKNATNAKGHASVKAGDTILFQWVPVPWPHPGPIVDYLANCNGD
CETVDKTTLEFFKIDGVGLLSGGDPGTWASDVLISNNNTWVVKIPDNLAP
GNYVLRHEIIALHSAGQANGAQNYPQCFNIAVSGSGSLQPSGVLGTDLYH
ATDPGVLINIYTSPLNYIIPGPTVVSGLPTSVAQGSSAATATASATVPGG
GSGPTSRTTTTARTTQASSRPSSTPPATTSAPAGGPTQTLYGQCGGSGYS
GPTRCAPPATCSTNPYYAQCLN
TreGlu6, Trichoderma reesei
(SEQ ID NO: 13)
AFSWKNVKLGGGGGFVPGIIFHPKTKGVAYARTDIGGLYRLNADDSWTAV
TDGIADNAGWHNWGIDAVALDPQDDQKVYAAVGMYTNSWDPSNGAIIRSS
DRGATWSFTNLPFKVGGNMPGRGAGERLAVDPANSNIIYFGARSGNGLWK
STDGGVTFSKVSSFTATGTYIPDPSDSNGYNSDKQGLMWVTFDSTSSTTG
GATSRIFVGTADNITASVYVSTNAGSTWSAVPGQPGKYFPHKAKLQPAEK
ALYLTYSWWPDAQLFRSTDSGTTWSPIWAWASYPTETYYYSISTPKAPWI
KNNFIDVTSESPSDGLIKRLGWMIESLEIDPTDSNHWLYGTGMTIFGGHD
LTNWDTRHNVSIQSLADGIEEFSVQDLASAPGGSELLAAVGDDNGFTFAS
RNDLGTSPQTVWATPTWATSTSVDYAGNSVKSVVRVGNTAGTQQVAISSD
GGATWSIDYAADTSMNGGTVAYSADGDTILWSTASSGVQRSQFQGSFASV
SSLPAGAVIASDKKTNSVFYAGSGSTFYVSKDTGSSFTRGPKLGSAGTIR
DIAAHPTTAGTLYVSTDVGIFRSTDSGTTFGQVSTALTNTYQIALGVGSG
SNWNLYAFGTGPSGARLYASGDSGASWTDIQGSQGFGSIDSTKVAGSGST
AGQVYVGTNGRGVFYAQGTVGGGTGGTSSSTKQSSSSTSSASSSTTLRSS
VVSTTRASTVTSSRTSSAAGPTGSGVAGHYAQCGGIGWTGPTQCVAPYVC
QKQNDYYYQCV
TreGlu7, Trichoderma reesei
(SEQ ID NO: 14)
HGQVQNFTINGQYNQGFILDYYYQKQNTGHFPNVAGWYAEDLDLGFISPD
QYTTPDIVCHKNAAPGAISATAAAGSNIVFQWGPGVWPHPYGPIVTYVVE
CSGSCTTVNKNNLRWVKIQEAGINYNTQVWAQQDLINQGNKWTVKIPSSL
RPGNYVFRHELLAAHGASSANGMQNYPQCVNIAVTGSGTKALPAGTPATQ
LYKPTDPGILFNPYTTITSYTIPGPALWQG
TreGlu8, Trichoderma reesei
(SEQ ID NO: 15)
GKIKYLGVAIPGIDFGCDIDGSCPTDTSSVPLLSYKGGDGAGQMKHFAED
DGLNVFRISATWQFVLNNTVDGKLDELNWGSYNKVVNACLETGAYCMIDM
HNFARYNGGIIGQGGVSDDIFVDLWVQIAKYYEDNDKIIFGLMNEPHDLD
IEIWAQTCQKVVTAIRKAGATSQMILLPGTNFASVETYVSTGSAEALGKI
TNPDGSTDLLYFDVHKYLDINNSGSHAECTTDNVDAFNDFADWLRQNKRQ
AIISETGASMEPSCMTAFCAQNKAISENSDVYIGFVGWGAGSFDTSYILT
LTPLGKPGNYTDNKLMNECILDQFTLDEKYRPTPTSISTAAEETATATAT
SDGDAPSTTKPIFREETASPTPNAVTKPSPDTSDSSDDDKDSAASMSAQG
LTGTVLFTVAALGYMLVAF
BsuGluC CBD, Bacillus subtilis
(SEQ ID NO: 16)
MKRSISIFITCLLITLLTMGGMIASPASAAGTKTPVAKNGQLSIKGTQLV
NRDGKAVQLKGISSHGLQWYGEYVNKDSLKWLRDDWGITVFRAAMYTADG
GYIDNPSVKNKVKEAVEAAKELGIYVIIDWHILNDGNPNQNKEKAKEFFK
EMSSLYGNTPNVIYEIANEPNGDVNWKRDIKPYAEEVISVIRKNDPDNII
IVGTGTWSQDVNDAADDQLKDANVMYALHFYAGTHGQFLRDKANYALSKG
APIFVTEWGTSDASGNGGVFLDQSREWLKYLDSKTISWVNWNLSDKQESS
SALKPGASKTGGWRLSDLSASGTFVRENILGTKDSTKDIPETPSKDKPTQ
ENGISVQYRAGDGSMNSNQIRPQLQIKNNGNTTVDLKDVTARYWYKAKNK
GQNFDCDYAQIGCGNVTHKFVTLHKPKQGADTYLELGFKNGTLAPGASTG
NIQLRLHNDDWSNYAQSGDYSFFKSNTFKTTKKITLYDQGKLIWGTEPN
BsuXyn3, Bacillus subtilis xylanase variant
(SEQ ID NO: 17)
ASTDYWQNWTFGGGIVNAVNGSGGNYSVNWSNTGNFVVGKGWTTGSPFRT
INYNAGVWAPNGNGYLTLYGWTRSPLIEYYVVDSWGTYRPTGTYKGTVKS
DGGTYDIYTTTRYNAPSIDGDDTTFTQYWSVRQSKRPTGSNATITFSNHV
NAWKSHGMNLGSNWAYQVMATEGYQSSGSSNVTVW
BsuXyn4, Bacillus subtilis xylanase variant
(SEQ ID NO: 18)
ASTDYWQNWTDGYGIVNAVNGSGGNYSVNWSNTGNFVVGKGWTTGSPFRT
INYNAGVWAPNGNGYLTLYGWTRSPLIEYYVVDSWGTYRPTGTYKGTVYS
DGGWYDIYTATRDNAPSIDGDFTTFTQYWSVRQSKRPTGSNATITFSNHV
NAWRSHGMDLGSNWAYQVMATEGYLSSGSSNVTVW
ApuXyn1, AUREOBASIDIUM PULLULANS, 343 AA
(SEQ ID NO: 19)
TPTDYSTSSYSKNQGLAQAWTSKGRQYIGTALTIRDDPVEQGIIQSRTDF
NSITPENAMKWESTEPQRNNFTFAGADAVADFADRYNKEMRCHTLVWHSQ
LPAWVSQGNFDNKTLISIMENHIKKVAGRYKNKCTHWDVVNEALNEDGTY
RSSVFYNTIGEAFIPIAFRFAEKYAGSKTKLYYNDYNLEYGSAKALGAQR
ILKLVQSYGVQIDGVGLQAHLSSEATASTGGGVTPDVQTLTNVLKLYTDL
GVEVAYTELDVRFTTPATDAKLKAQADAYARVVQSCINVKRCVGITVWGV
SDKYSWIPGVFPTEGAALLWDENFNKKPAYSSVLKTIQSFRKS
TlaXyn1, THERMOMYCES LANUGINOSUS, 225 AA
(SEQ ID NO: 20)
AFPAGNATELEKRQTTPNSEGWHDGYYYSWWSDGGAQATYTNLEGGTYEI
SWGDGGNLVGGKGWNPGLNARAIHFEGVYQPNGNSYLAVYGWTRNPLVEY
YIVENFGTYDPSSGATDLGTVECDGSIYRLGKTTRVNAPSIDGTQTFDQY
WSVRQDKRTSGTVQTGCHFDAWARAGLNVNGDHYYQIVATEGYFSSGYAR
ITVADVGMVGFTPVALAALAATGAL
TauXyn1, THERMOASCUS AURANTIACUS; 310 AA
(SEQ ID NO: 21)
SPILEERQAAQSVDQLIKARGKVYFGVATDQNRLTTGKNAAIIQADFGQV
TPENSMKWDATEPSQGNFNFAGADYLVNWAQQNGKLIRGHTLVWHSQLPS
WVSSITDKNTLTNVMKNHITTLMTRYKGKIRAWDVVNEAFNEDGSLRQTV
FLNVIGEDYIPIAFQTARAADPNAKLYINDYNLDSASYPKTQAIVNRVKQ
WRAAGVPIDGIGSQTHLSAGQGAGVLQALPLLASAGTPEVAITELDVAGA
SPTDYVNVVNACLNVQSCVGITVWGVADPDSWRASTTPLLFDGNFNPKPA
YNAIVQDLQQ,
SthXyn1, S. THERMOVIOLACEUS, 295 AA
(SEQ ID NO: 22)
DTITSNQTGTHNGYFYSFWTDAPGTVTMNTGAGGNYSTQWSNTGNFVAGK
GWATGGRRTVTYSGTFNPSGNAYLALYGWSQNPLVEYYIVDNWGTYRPTG
TYKGTVDSDGGTYDIYMTTRYNAPSIEGTKTFNQYWSVRQNKRTGGTITT
GNHFDAWAAHGMPLGTFNYMILATEGYQSSGSSNITVGDSGGDNGGGGGG
GGGGGNTGGCTATLSAGEQWSDRYNLNVSVSGSDNWTVTMRVPAPEKVMA
TWNVTASYPDAQTLVARPNGNGNNWGVTIQKNGSTTWPTVSCSVG
TfuXyn1, THERMOBIFIDA FUSCA, 296 AA
(SEQ ID NO: 23)
AVTSNETGYHDGYFYSFWTDAPGTVSMELGPGGNYSTSWRNTGNFVAGKG
WATGGRRTVTYSASFNPSGNAYLTLYGWTRNPLVEYYIVESWGTYRPTGT
YMGTVTTDGGTYDIYKTTRYNAPSIEGTRTFDQYWSVRQSKRTSGTITAG
NHFDAWARHGMHLGTHDYMIMATEGYQSSGSSNVTLGTSGGGNPGGGNPP
GGGNPPGGGGCTATLSAGQQWNDRYNLNVNVSGSNNWTVTVNVPWPARII
ATWNIHASYPDSQTLVARPNGNGNNWGMTIMHNGNWTWPTVSCSAN
FoxXyn2*, FUSARIUM OXYSPORUM
(SEQ ID NO: 24)
MKLSSFLYTASLVAAIPTAIEPRQASDSINKLIKNKGKLYYGTITDPNLL
GVAKDTAIIKADFGAVTPENSMKWDATEPSQGKFNFGSFDQVVNFAQQNG
LKVRGHTVVWHSQLPQWVKNINDKATLTKVIENHVTNVVGRYKGKIYAWD
VVNEIFDWDGTLRKDSHFNNVFGNDDYVGIAFRAARKADPNAKLYINDYS
LDSGSASKVTKGMVPSVKKWLSQGVPVDGIGSQTHLDPGAAGQIQGALTA
LANSGVKEVAITELDIRTAPANDYATVTKACLNVPKCIGITVWGVSDKNS
WRKEHDSLLFDANYNPKAAYTAVVNALR
FveXyn4*, FUSARIUM VERTICILLOIDES
(SEQ ID NO: 25)
QAADSINKLIKNKGKLYYGTITDPNLLGVAKDTAIIKADFGAVTPENSGK
WDATEPSQGKFNFGSFDQVVNFAQQNGLKVRGHTLVWHSQLPQWVKNIND
KATLTKVIENHVTQVVGRYKGKIYAWDVVNEIFEWDGTLRKDSHFNNVFG
NDDYVGIAFRAARKADPNAKLYINDYSLDSGSASKVTKGMVPSVKKWLSQ
GVPVDGIGSQTHLDPGAAGQIQGALTALANSGVKEVAITELDIRTAPAND
YATVTKACLNVPKCIGITVWGVSDKNSWRKEHDSLLFDANYNPKPAYTAW
NALR

1. An enzyme exhibiting endo-1,443-xylanase activity, which enzyme comprises an amino acid sequence having at least 80% identity with any one selected from SEQ ID NO:1, SEQ ID NO:2, SEQ ID NO:3, SEQ ID NO:4, SEQ ID NO:5, SEQ ID NO:6, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25, or any functional fragment thereof.

2. (canceled)

3. An enzyme exhibiting endo-1,3(4)-(3-glucanase activity, which enzyme comprises an amino acid sequence having at least 80% identity with any one selected from SEQ ID NO:7, SEQ ID NO:8, SEQ ID NO:9, SEQ ID NO:10, and SEQ ID NO:11, SEQ ID NO:12, SEQ ID NO:13, SEQ ID NO:14, SEQ ID NO:15, SEQ ID NO:16, or any functional fragment thereof.

4. (canceled)

5. The enzyme according to claim 1, wherein said enzyme has at least 81, 82, 83, 84, 85, 86, 87, 88, 89, 90, 91, 92, 93, 94, 95, 96, 97, 98 or 99% identity with any one amino acid sequence selected from SEQ ID NO: 1-25, or any functional fragment thereof.

6-9. (canceled)

10. The enzyme according to any one of claim 1 or 3, which enzyme consists of the amino acid sequence identified by any one of SEQ ID NO: 1-25, or any functional fragment thereof.

11. A DNA construct comprising a DNA sequence encoding an enzyme according to claim 1.

12. A recombinant expression vector comprising a DNA construct according to claim 11.

13. A cell that has been transformed with a DNA construct of claim 11 or the vector of claim 12.

14. (canceled)

15. A composition comprising an enzyme exhibiting endo-1,4-6-xylanase activity according to claim 1 in combination with any one or more 6-glucanase.

16. The composition according to claim 15, wherein said one or more 6-glucanase is an enzyme exhibiting endo-1,3(4)-6-glucanase activity according to claim 3.

17. (canceled)

18. (canceled)

19. The composition according to claim 15, wherein said endo-1,3(4)-6-glucanase activity and said endo-1,4-6-xylanase activity are derived from at least two different enzymes, such as at least two different enzymes from two different species.

20. The composition according to claim 15, comprising a combination of at least two enzymes, said two enzymes, or two enzymes with an amino acid sequence having at least 80% sequence identity with the respective SEQ ID, or any functional fragment thereof, being selected from the list consisting of
SEQ ID NO:1 and SEQ ID NO:7;
SEQ ID NO:2 and SEQ ID NO:7;
SEQ ID NO:3 and SEQ ID NO:7;
SEQ ID NO:4 and SEQ ID NO:7;
SEQ ID NO:5 and SEQ ID NO:7;
SEQ ID NO:6 and SEQ ID NO:7;
SEQ ID NO:17 and SEQ ID NO:7;
SEQ ID NO:18 and SEQ ID NO:7;
SEQ ID NO:19 and SEQ ID NO:7;
SEQ ID NO:20 and SEQ ID NO:7;
SEQ ID NO:21 and SEQ ID NO:7;
SEQ ID NO:22 and SEQ ID NO:7;
SEQ ID NO:23 and SEQ ID NO:7;
SEQ ID NO:24 and SEQ ID NO:7;
SEQ ID NO:25 and SEQ ID NO:7;
SEQ ID NO:1 and SEQ ID NO:8;
SEQ ID NO:2 and SEQ ID NO:8;
SEQ ID NO:3 and SEQ ID NO:8;
SEQ ID NO:4 and SEQ ID NO:8;
SEQ ID NO:5 and SEQ ID NO:8;
SEQ ID NO:6 and SEQ ID NO:8;
SEQ ID NO:17 and SEQ ID NO:8;
SEQ ID NO:18 and SEQ ID NO:8;
SEQ ID NO:19 and SEQ ID NO:8;
SEQ ID NO:20 and SEQ ID NO:8;
SEQ ID NO:21 and SEQ ID NO:8;
SEQ ID NO:22 and SEQ ID NO:8;
SEQ ID NO:23 and SEQ ID NO:8;
SEQ ID NO:24 and SEQ ID NO:8;
SEQ ID NO:25 and SEQ ID NO:8;
SEQ ID NO:1 and SEQ ID NO:9;
SEQ ID NO:2 and SEQ ID NO:9;
SEQ ID NO:3 and SEQ ID NO:9;
SEQ ID NO:4 and SEQ ID NO:9;
SEQ ID NO:5 and SEQ ID NO:9;
SEQ ID NO:6 and SEQ ID NO:9;
SEQ ID NO:17 and SEQ ID NO:9;
SEQ ID NO:18 and SEQ ID NO:9;
SEQ ID NO:19 and SEQ ID NO:9;
SEQ ID NO:20 and SEQ ID NO:9;
SEQ ID NO:21 and SEQ ID NO:9;
SEQ ID NO:22 and SEQ ID NO:9;
SEQ ID NO:23 and SEQ ID NO:9;
SEQ ID NO:24 and SEQ ID NO:9;
SEQ ID NO:25 and SEQ ID NO:9;
SEQ ID NO:1 and SEQ ID NO:10;
SEQ ID NO:2 and SEQ ID NO:10;
SEQ ID NO:3 and SEQ ID NO:10;
SEQ ID NO:4 and SEQ ID NO:10;
SEQ ID NO:5 and SEQ ID NO:10;
SEQ ID NO:6 and SEQ ID NO:10;
SEQ ID NO:17 and SEQ ID NO:10;
SEQ ID NO:18 and SEQ ID NO:10;
SEQ ID NO:19 and SEQ ID NO:10;
SEQ ID NO:20 and SEQ ID NO:10;
SEQ ID NO:21 and SEQ ID NO:10;
SEQ ID NO:22 and SEQ ID NO:10;
SEQ ID NO:23 and SEQ ID NO:10;
SEQ ID NO:24 and SEQ ID NO:10;
SEQ ID NO:25 and SEQ ID NO:10;
SEQ ID NO:1 and SEQ ID NO:11;
SEQ ID NO:2 and SEQ ID NO:11;
SEQ ID NO:3 and SEQ ID NO:11;
SEQ ID NO:4 and SEQ ID NO:11;
SEQ ID NO:5 and SEQ ID NO:11;
SEQ ID NO:6 and SEQ ID NO:11;
SEQ ID NO:17 and SEQ ID NO:11;
SEQ ID NO:18 and SEQ ID NO:11;
SEQ ID NO:19 and SEQ ID NO:11;
SEQ ID NO:20 and SEQ ID NO:11;
SEQ ID NO:21 and SEQ ID NO:11;
SEQ ID NO:22 and SEQ ID NO:11;
SEQ ID NO:23 and SEQ ID NO:11;
SEQ ID NO:24 and SEQ ID NO:11;
SEQ ID NO:25 and SEQ ID NO:11;
SEQ ID NO:1 and SEQ ID NO:12;
SEQ ID NO:2 and SEQ ID NO:12;
SEQ ID NO:3 and SEQ ID NO:12;
SEQ ID NO:4 and SEQ ID NO:12;
SEQ ID NO:5 and SEQ ID NO:12;
SEQ ID NO:6 and SEQ ID NO:12;
SEQ ID NO:17 and SEQ ID NO:12;
SEQ ID NO:18 and SEQ ID NO:12;
SEQ ID NO:19 and SEQ ID NO:12;
SEQ ID NO:20 and SEQ ID NO:12;
SEQ ID NO:21 and SEQ ID NO:12;
SEQ ID NO:22 and SEQ ID NO:12;
SEQ ID NO:23 and SEQ ID NO:12;
SEQ ID NO:24 and SEQ ID NO:12;
SEQ ID NO:25 and SEQ ID NO:12;
SEQ ID NO:1 and SEQ ID NO:13;
SEQ ID NO:2 and SEQ ID NO:13;
SEQ ID NO:3 and SEQ ID NO:13;
SEQ ID NO:4 and SEQ ID NO:13;
SEQ ID NO:5 and SEQ ID NO:13;
SEQ ID NO:6 and SEQ ID NO:13;
SEQ ID NO:17 and SEQ ID NO:13;
SEQ ID NO:18 and SEQ ID NO:13;
SEQ ID NO:19 and SEQ ID NO:13;
SEQ ID NO:20 and SEQ ID NO:13;
SEQ ID NO:21 and SEQ ID NO:13;
SEQ ID NO:22 and SEQ ID NO:13;
SEQ ID NO:23 and SEQ ID NO:13;
SEQ ID NO:24 and SEQ ID NO:13;
SEQ ID NO:25 and SEQ ID NO:13;
SEQ ID NO:1 and SEQ ID NO:14;
SEQ ID NO:2 and SEQ ID NO:14;
SEQ ID NO:3 and SEQ ID NO:14;
SEQ ID NO:4 and SEQ ID NO:14;
SEQ ID NO:5 and SEQ ID NO:14;
SEQ ID NO:6 and SEQ ID NO:14;
SEQ ID NO:17 and SEQ ID NO:14;
SEQ ID NO:18 and SEQ ID NO:14;
SEQ ID NO:19 and SEQ ID NO:14;
SEQ ID NO:20 and SEQ ID NO:14;
SEQ ID NO:21 and SEQ ID NO:14;
SEQ ID NO:22 and SEQ ID NO:14;
SEQ ID NO:23 and SEQ ID NO:14;
SEQ ID NO:24 and SEQ ID NO:14;
SEQ ID NO:25 and SEQ ID NO:14;
SEQ ID NO:1 and SEQ ID NO:15;
SEQ ID NO:2 and SEQ ID NO:15;
SEQ ID NO:3 and SEQ ID NO:15;
SEQ ID NO:4 and SEQ ID NO:15;
SEQ ID NO:5 and SEQ ID NO:15;
SEQ ID NO:6 and SEQ ID NO:15;
SEQ ID NO:17 and SEQ ID NO:15;
SEQ ID NO:18 and SEQ ID NO:15;
SEQ ID NO:19 and SEQ ID NO:15;
SEQ ID NO:20 and SEQ ID NO:15;
SEQ ID NO:21 and SEQ ID NO:15;
SEQ ID NO:22 and SEQ ID NO:15;
SEQ ID NO:23 and SEQ ID NO:15;
SEQ ID NO:24 and SEQ ID NO:15;
SEQ ID NO:25 and SEQ ID NO:15;
SEQ ID NO:1 and SEQ ID NO:16;
SEQ ID NO:2 and SEQ ID NO:16;
SEQ ID NO:3 and SEQ ID NO:16;
SEQ ID NO:4 and SEQ ID NO:16;
SEQ ID NO:5 and SEQ ID NO:16;
SEQ ID NO:6 and SEQ ID NO:16;
SEQ ID NO:17 and SEQ ID NO:16;
SEQ ID NO:18 and SEQ ID NO:16;
SEQ ID NO:19 and SEQ ID NO:16;
SEQ ID NO:20 and SEQ ID NO:16;
SEQ ID NO:21 and SEQ ID NO:16;
SEQ ID NO:22 and SEQ ID NO:16;
SEQ ID NO:23 and SEQ ID NO:16;
SEQ ID NO:24 and SEQ ID NO:16; and
SEQ ID NO:25 and SEQ ID NO:16.

21-24. (canceled)

25. The composition according claim 15, comprising any one or more further enzyme.

26. The composition according to claim 25, wherein said one or more further enzyme is selected from list consisting of a xylanase classified in EC 3.2.1.32, EC 3.2.1.136, or EC 3.2.1.156, a cellulase, a laminarinase, an endo-1,5-a-L-arabinanase, a beta-D-glucoside glucohydrolase, a (3-Xylosidase, a cellobiohydrolase, a glucan 1,4-beta-glucosidase, a xyloglucan-specific exo-beta-1,4-glucanase and an a-N-Arabinofuranosidase.

27-34. (canceled)

35. A method of altering filterability of a starch comprising material, said method comprising the step of treating said starch comprising material with enzyme according to claim 1 or a composition according to claim 15.

36. A method of reducing pressure built up during lautering in a brewing application, said method comprising the step of treating a brewing mash with enzyme according to claim 1 or a composition according to claim 15.

37. A method for the production of a food, feed, or beverage product, such as an alcoholic or non-alcoholic beverage, such as a cereal- or malt-based beverage like beer or whiskey, said method comprising the step of treating a starch comprising material with enzyme according to claim 1 or a composition according to claim 15.

38. A method for the production of a brewing mash, said method comprising the step of treating a starch comprising material with enzyme according to claim 10 or a composition according to claim 15.

39. A method for the production of a first—or second-generation biofuel, such as bioethanol, said method comprising the step of treating a starch comprising material with an enzyme according to claim 1 or a composition according to claim 15.

40. (canceled)

41. (canceled)
